SEQUENCE LISTING (9 sequences)

SEQ ID NO: 1 - protein, 454 aa, Homo sapiens
MARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 2 - protein, 392 aa, Homo sapiens
MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTNVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 3 - protein, 319 aa, Homo sapiens
MARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRR

SEQ ID NO: 4 - protein, 267 aa, Homo sapiens
MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTNVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTR

SEQ ID NO: 5 - protein, 135 aa, Homo sapiens
GQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 6 - protein, 125 aa, Homo sapiens
QADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 7 - protein, 32 aa, Artificial Sequence
  Peptide
VPAKYSPLSVLTIEPDGSIAYKEYEDMIATKC

SEQ ID NO: 8 - protein, 16 aa, Artificial Sequence
  Peptide
KKPLGPASFNLSEYFC

SEQ ID NO: 9 - protein, 18 aa, Artificial Sequence
  Peptide
SEVTASSSKHSGPENNQC

(19) United States
(12) Patent Application Publication
(10) Pub. No.: US 2020/0041523 A1
(43) Pub. Date: Feb. 6, 2020

(54) GAMETE-SECRETED GROWTH FACTORS

(71) Applicants: NEWSOUTH INNOVATIONS PTY LIMITED, Sydney, NSW (AU); HUDSON INSTITUTE OF MEDICAL RESEARCH, Clayton, VIC (AU)

(72) Inventors: Robert Bruce GILCHRIST, Maroubra, NSW (AU); Karen CHAN, Reservoir, VIC (AU); William Leigh LEDGER, Clovelly, NSW (AU); David Mark MILNE-ROBERTSON, Helensburgh, NSW (AU); Angelique Helena RIEPSAMEN, Randwick, NSW (AU)

(21) Appl. No.: 16/482,834

(22) PCT Filed: Feb. 1, 2018
(86) PCT No.: PCT/AU2018/050064

§ 371 Date: Aug. 1, 2019

(30) Foreign Application Priority Data
Feb. 1, 2017 (AU) 2017900297

(51) Int. Cl. G01N 33/68; G01N 33/563; C07K 16/22

(52) U.S. Cl. CPC G01N 33/689; G01N 33/563; G01N 2800/367; G01N 2333/495; C07K 16/22

(57) ABSTRACT

The present invention relates to diagnostic markers of fertility, reproductive dysfunction and infertility management. In particular, the invention relates a method for predicting the fertility potential of a subject, the method comprising determining the level of one or more of GDF9, BMP15 and/or cumulin in the subject.

### FIELD

The present invention relates to diagnostic markers of fertility, reproductive dysfunction and infertility management. In particular, the invention relates to biomarkers of oocyte quantity and quality, sperm quality, and fertility potential.

### BACKGROUND

Managing fertility is a major global health challenge, whether it is the control of fertility through contraception or the diagnosis and treatment of reproductive diseases and infertility. One in six couples worldwide experience infertility and ˜3-4% of all babies born in most Western countries are the result of advanced reproductive technologies, such as in vitro fertilisation (IVF). Key to the modern practice of IVF are diagnostics, in particular the measurement of a large array of hormones from blood samples, especially from the female partner. Hormone measures are required to accurately diagnose reproductive dysfunction and disease to determine the best course of subsequent treatment. In addition, during the course of treatment (e.g. IVF), repeated blood samples are taken to monitor the patient's response to drugs, in particular to the administration of follicle stimulating hormone (FSH).

Important hormones that are routinely measured from blood samples before and/or during an ovarian hyperstimulation cycle for IVF include: anti-Müllerian hormone (AMH), oestradiol, progesterone, FSH and luteinising hormone (LH), amongst others. AMH provides an indication of the number of small antral follicles, and hence is commonly used clinically as an indirect estimate of ovarian reserve (or future fertility potential). Oestradiol provides a reliable measure of the growth of ovarian follicles in response to exogenous FSH. Both AMH and oestradiol are produced by the mural granulosa cells of the ovarian follicle.

Despite its widespread use, IVF remains inefficient with only a 17.9% success rate (live birth/IVF cycle initiated), and it is expensive. Oocyte quantity and oocyte quality are the key rate-limiting factors in IVF success. This is clear from the fact that oocyte quantity and quality decline dramatically in women at around forty years of age, causing declining fertility and the eventual onset of menopause.

Notably, there are no direct measures of oocyte quantity and quality. This represents one of the greatest unmet clinical needs in IVF, as the success of the technology relies on the generation of supernumerary oocytes/embryos (e.g. 5-15/IVF cycle) and the subsequent transfer of a single embryo in consecutive cycles, or multiple embryos, back to the patient. One measure of the potential number of oocytes that might be retrieved in an IVF cycle is “antral follicle count (AFC)”, which is determined by vaginal ultrasound. Combining AFC with serum AMH values provides a clinically useful estimate of potential oocyte quantity.

Accordingly, there remains a need for tests to assist clinicians in patient management and treatment of reproductive diseases including infertility.

### SUMMARY

The inventors have determined that assays for measuring the level of GDF9, BMP15 and/or cumulin in patients are useful as diagnostic and/or predictive markers for men and women with infertility or undergoing fertility treatment such as IVF treatment. The inventors have demonstrated that the assays provide additional and complementary information to the clinician in his/her diagnosis and patient management, and that the assays may be used alone or in addition to existing diagnostic tests.

Accordingly, in one aspect there is provided a method for predicting the fertility potential of a subject, the method comprising determining the level of one or more of GDF9, BMP15 and/or cumulin in the subject.

In one embodiment, the level of GDF9, BMP15 and/or cumulin is indicative of oocyte quality and/or oocyte quantity.

In another embodiment, the level of GDF9, BMP15, and/or cumulin is indicative of sperm quality. As understood in the art, sperm quality can be measured by sperm quantity, motility and morphology. In one embodiment, the sperm quality may be sperm motility or sperm abnormality.

The methods and assays developed by the present inventors are useful in determining the likelihood that a pregnancy will result in a woman attempting to conceive naturally, or particularly during fertility treatment. Thus, in another aspect, there is provided a method of predicting pregnancy success in a subject, the method comprising determining the level of one or more of GDF9, BMP15 and/or cumulin in the subject.

In one embodiment, a low level of GDF9, BMP15 and/or cumulin in the subject compared to a reference level is indicative of low fertility potential and/or is predictive of a low chance of pregnancy success.

The inventors also determined that levels of GDF9, BMP15 and/or cumulin in a subject are indicative of reproductive disease. Thus, in a further aspect there is provided a method of diagnosing or predicting a reproductive disease in a subject, the method comprising determining the level of one or more of GDF9, BMP15 and/or cumulin in the subject.

In one embodiment of the methods described herein, the subject is undergoing fertility treatment. In one specific example, the subject is undergoing a fertility treatment selected from Ovulation Induction (OI), Intra-Uterine Insemination (IUI), In Vitro Fertilisation (IVF) treatment, Intra-cytoplasmic Sperm Injection (ICSI), In Vitro Maturation (IVM); frozen embryo transfer (FET) and/or other assisted reproductive technology.

In one embodiment, the reproductive disease is premature menopause, polycystic ovaries (PCO), polycystic ovarian syndrome (PCOS) or endometriosis.

In yet another embodiment of the method or assay described herein, the level of GDF9, BMP15 and/or cumulin is determined in a sample obtained from the subject. In one embodiment, the sample comprises serum, plasma, urine, semen, follicular fluid, somatic cells, culture medium conditioned by an oocyte or embryo, and/or biological material collected during IVF or ICSI treatment.

In one embodiment, the follicular fluid and/or somatic cells are collected prior to treatment, or during IVF or ICSI treatment.

In one embodiment, the method comprises testing for another marker, such as a marker known to be associated with fertility and/or reproductive disease. In one particular embodiment, the subject is female and the method further comprises determining the level of anti-Mullerian hormone (AMH) in a sample from the subject.

As understood by the person skilled in the art, the methods and assays described herein may be performed by comparing levels of markers in a subject sample to a reference sample, or a prepared data set, for example as prepared from a reference population. Thus, in one embodiment, the method comprises comparing the level of GDF9, BMP15 and/or cumulin in the subject with the level of GDF9, BMP15 and/or cumulin in a reference sample or reference population.

In one embodiment, a higher level of GDF9, BMP15 and/or cumulin in the subject when compared to the level of GDF9, BMP15 and/or cumulin in the reference sample or reference population indicates a higher number of oocytes can be retrieved from the subject.

In another embodiment, the subject is a PCOS patient undergoing OI, IUI, ICSI, or IVF, and the method comprises determining the level of BMP15.

In yet another embodiment, a lower level of GDF9 in a male subject when compared to the level of GDF9 in a reference sample or reference population is indicative of reduced sperm motility and/or indicative of abnormal sperm morphology.

In another aspect, there is further provided a method of determining the level of GDF9, BMP15 and/or cumulin in a subject sample, the method comprising determining the level of GDF9, BMP15 and/or cumulin in the sample by contacting the sample with an anti-GDF9 antibody, an anti-BMP15 antibody and/or an anti-cumulin antibody.

In one embodiment, determining the level of GDF9, BMP15 and/or cumulin comprises detecting a complex of the anti-GDF9 antibody, an anti-BMP15 antibody and/or an anti-cumulin antibody with the GDF9, BMP15 and/or cumulin. In one embodiment, the antibody is detectably labelled.

The subject sample may be any suitable biological sample in which GDF9, BMP15 and/or cumulin may be detected. The sample may be obtained when the patient is healthy, prior to or during fertility treatment, and/or following a diagnosis of reproductive disease. In one embodiment, the sample is serum, plasma, urine, semen, follicular fluid, somatic cells, culture medium conditioned by an oocyte or embryo, and/or biological material collected during IVF treatment.

In one particular embodiment, the present invention provides a method of determining the reproductive quality of a subject's oocyte/embryo, the method comprising determining the level of one or more of GDF9, BMP15 and/or cumulin in the subject.

It is well known that the quality of a subject's oocytes/embryos has a direct correlation with the success of, for example IVF. Current selection procedures are mostly entirely based on morphological evaluation of the embryo at different timepoints during development and particularly an evaluation at the time of transfer using a standard stereomicroscope. It is known that oocyte/embryo quality may be assessed by any number techniques including measuring (i) the cell division time period for at least one cell division, (ii) the time period of inter-division period, (iii) the time period of cellular movement in inter-division period, and/or (iv) the extent of cellular movement in inter-division period. However, the present invention assesses the quality of the oocyte/embryo by determining the level of one or more of GDF9, BMP15 and/or cumulin as compared to known standards.

In one particular embodiment, the culture medium conditioned by an oocyte or embryo, follicular fluid and/or somatic cells are collected during IVF treatment.

In one embodiment, the level of GDF9, BMP15 and/or cumulin is determined by ELISA assay.

In another embodiment, the method comprises determining the level of cumulin by contacting the sample with an anti-GDF9 antibody and an anti-BMP15 antibody.

In another aspect, there is provided method of performing Ovulation Induction (OI), In Vitro Fertilisation (IVF) treatment, Intra-cytoplasmic Sperm Injection (ICSI) treatment, Intrauterine Insemination (IUI), In Vitro Maturation (IVM); frozen embryo transfer (FET) or other assisted reproductive technology on a patient, the method comprising:
i) determining the level of GDF9, BMP15 and/or cumulin in the patient, and
ii) modifying the course of treatment of the 01, IVF, ICSI, IUI, IVM, FET or other assisted reproductive technology based on the level of GDF9, BMP15 and/or cumulin in the patient.

In one embodiment, the level of GDF9, BMP15 and/or cumulin is determined in a patient sample.

In another embodiment, the method comprises obtaining the sample from the patient.

In an embodiment, the method comprises determining the level GDF9, BMP15 and/or cumulin in a sample obtained from the patient.

In one embodiment, the sample is serum, plasma, urine, semen, follicular fluid, somatic cells, culture medium conditioned by an oocyte or embryo, and/or biological material collected during IVF treatment.

In one particular embodiment, the follicular fluid and/or somatic cells are collected during IVF treatment.

In another embodiment, the level of GDF9, BMP15 and/or cumulin is determined by ELISA assay.

In one embodiment of the methods described herein, the method further comprises directing treatment based on the level of GDF9, BMP15 and/or cumulin in a subject or patient sample. For example, directing treatment may comprise initiating Ovulation Induction (OI), In Vitro Fertilisation (IVF) treatment, Intra-cytoplasmic Sperm Injection (ICSI) treatment, Intrauterine Insemination (IUI), In Vitro Maturation (IVM); frozen embryo transfer (FET) or other assisted reproductive technology on the subject or patient.

In one embodiment, directing treatment comprises altering a patient hormonal regime during fertility treatment. In another embodiment, directing treatment comprises referring the subject or patient for additional diagnostic examination. In one particular embodiment, the subject or patient is a male and is referred for full-semen analysis and/or additional blood tests for male-factor infertility.

In yet another embodiment, directing treatment comprises altering laboratory procedures for oocyte insemination, for example, utilising intra-cytoplasmic insemination instead of IVF for men with aberrant levels of GDF9, BMP15 and/or cumulin when compared to a reference level.

In another embodiment, directing treatment comprises performing an additional investigation on the subject or patient, such as ultrasound, or an additional treatment on the subject such laparascopic surgery for endometriosis.

In another aspect, there is provided a kit, assay or device for determining the level of GDF9, BMP15 and/or cumulin in a patient sample, the kit assay or device comprising one or more reagents to detect GDF9, BMP15 and/or cumulin in the sample, wherein the sample is selected from serum, plasma, urine, semen, follicular fluid, somatic cells, and/or biological material collected during IVF treatment.

In yet another aspect, there is provided a kit, assay or device for assessing fertility comprising:
(i) one or more reagents to detect GDF9, BMP15 and/or cumulin in a biological sample selected from serum, plasma, follicular fluid and somatic cells; and
(ii) instructions for use.

In one embodiment, the one or more reagents comprises an anti-GDF9 antibody, an anti-BMP15 antibody and/or an anti-cumulin antibody.

In one particular embodiment, the biological sample is serum or plasma.

While the skilled person will appreciate there are a number of assays and techniques available for the detection of polypeptide markers in a patient sample, in one embodiment, the assay is an ELISA assay.

In yet another embodiment, the assay further comprises a reference sample.

In one embodiment, the kit, assay or device comprises an antibody that is detectably labelled.

In yet another embodiment, the device is a point-of-care device such as a lateral flow immunoassay device (immunochromatographic test strips).

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE FIGURES

FIG. 1. GDF9 ELISA. A GDF9 ELISA was developed to measure the amount of GDF9 in the HEK-283T conditioned medium. Recombinant mouse GDF9 (●) was used as a standard, and the specificity of the assay was assessed using a range of TGF-β family members; wild-type human GDF9 (♦), human GDF9 L40V (⋄), human BMP15 (□), human activin A (◯), and human TGF-β3 (▾). Dilutions of concentrated media from cells transfected with empty vector, pcDNA3.1 (r), were included as controls. The ELISA has a specificity of less than 0.1% in relation to the above TGF-β family members, with a sensitivity of 0.2 ng/ml. Values represent ±SEM in duplicate, from a representative experiment.

FIG. 2. Specificity of GDF9, BMP15 and Cumulin ELISAs. Dose response curves of reference preparations in the various ELISAs; (A) GDF9, (B) BMP15, and (C) Cumulin. (A) GDF9 ELISA: Coat 72B-Biot 53-1; recombinant mouse GDF9 (●) from R&D Systems, high molecular weight (HMW) recombinant human BMP15 (♦), recombinant human cumulin (▴). (B) GDF9 ELISA: Coat 72B-Biot 53-1; recombinant mouse GDF9 (●) from R&D Systems, high molecular weight (HMW) recombinant human BMP15 (♦), recombinant human cumulin (▴). (C) CUMULIN ELISA: Coat 72B-Biot 28A; recombinant human cumulin (◯), recombinant mouse GDF9 from R&D Systems (♦), high molecular weight (HMW) recombinant human BMP15 (●), high molecular weight (HMW) recombinant human GDF9 (▴).

FIG. 3. Dose response curves of serum and human BMP15 reference preparations in BMP15 ELISA under serum assay conditions. On the X-axis the BMP15 reference preparation is presented as ng/ml. Dose response dilutions of serum QCs are presented as doubling doses arbitrarily positioned on the X axis.

FIG. 4. Serum biomarker levels in patients (non-PCO(S) and PCO(S)), with individual patients grouped relative to number of oocytes retrieved during an IVF cycle (0-5, 6-10, 11-15 and >16 oocytes per pick-up); (A) GDF9, (B) BMP15, (C) AMH.

FIG. 5. Optimising the GDF9 ELISA in application to serum. The effects of the addition of 1M NaCl and male serum to the GDF9 ELISA. (A) Buffer A: 100 mM Tris/HCl pH8.0, 0.5% BSA, 1M NaCl, 1% Tween 20, No Male serum; (B) Buffer B+male serum: 100 mM Tris/HCl pH8.0, 0.5% BSA 0.154M NaCl, 0.1% Tween 20, with Male serum; (C) Buffer A+male serum: 100 mM Tris/HCl pH8.0, 0.5% BSA, 1M NaCl, 1% Tween 20, with Male serum.

FIG. 6. Correlation of serum GDF9 and BMP15 levels in all patients (non-PCO(S) and PCO(S)). Dots represent individual patients.

FIG. 7. Serum biomarker levels in patients (non-PCO(S) and PCO(S)), with individual patients grouped relative to number of oocytes (<10 and 10 oocytes) retrieved during an IVF cycle; (A) GDF9, (B) BMP15, (C) AMH, (D) BMP15:GDF9.

FIG. 8. Serum biomarker levels in non-PCO(S) patients relative to number of oocytes retrieved during an IVF cycle; (A) GDF9, (B) BMP15, (C) AMH. Dots represent individual patients.

FIG. 9. Serum biomarker levels in PCO(S) patients relative to number of oocytes retrieved during an IVF cycle; (A) GDF9, (B) BMP15, (C) AMH. Dots represent individual patients.

FIG. 10. Serum biomarker levels in non-PCO(S) and PCO(S) patients combined relative to number of oocytes retrieved during an IVF cycle; (A) GDF9, (B) BMP15, (C) AMH. Dots represent individual patients.

FIG. 11. Serum biomarker levels in non-PCO(S) and PCO(S) patients combined relative to number of oocytes retrieved during an IVF cycle (A, C), and associated ROC curves (B). (A, B) GDF9:AMH ratio, (C) BMP15:GDF9 ratio.

FIG. 12. Serum BMP15:AMH ratios in non-PCO(S) and PCO(S) patients combined relative to number of oocytes retrieved during an IVF cycle (A) and associated ROC curves (B).

FIG. 13. Serum biomarker levels in patients clinically assessed for endometriosis. (A) GDF9 in all patients; (B) GDF9 in patients with detectable GDF9; (C) BMP15 in all patients; (D) BMP15 in patients with detectable BMP15; (E) BMP15:GDF9 ratio in patients with detectable BMP15 and GDF9.

FIG. 14. ROC curve analyses for serum GDF9, BMP15 and GDF9:BMP15 ratio in patients clinically assessed for endometriosis.

FIG. 15. Serum biomarker levels relative to patient age; (A) GDF9, (B) BMP15, (C) AMH. Dots represent individual patients.

FIG. 16. Serum BMP15 levels in women throughout an antagonist stimulation cycle for IVF. (A) Patient cycle day relative to a baseline blood prior to stimulation. (B) Individual patients showing consecutive blood samples (days) within one stimulation cycle for IVF. Dashed line is the limit of detection of the ELISA.

FIG. 17. Male serum GDF9 levels relative to evidence of male-factor infertility. GDF9 in male serum with evidence of male-factor fertility.

FIG. 18. Development of a protocol for extraction of GDF9 and BMP15 from the surface of human cumulus cells. The effect of salt concentration on the extraction of GDF9 (A, B) and BMP15 (C) from human cumulus cells collected from patients undergoing infertility treatment using ICSI (intra-cytoplasmic sperm injection). (A, B) Dose response curves of cumulus cell extracts in the GDF9 ELISA. Cumulus cells extracted with salt concentrations of 1.5-2M gave maximal responses compared to 0.125M and 1M NaCl. (C) BMP15 levels are expressed relative to cumulus cell DNA content. A salt concentration of 1.5M was chosen for subsequent expts for both the GDF9 and BMP15 ELISAs.

FIG. 19. GDF9 (A) and BMP15 (B) ELISA dose response curves with recombinant human GDF9 and BMP15 as reference preparations, and extracts of human cumulus and granulosa cells. Non-parallelism was observed between GDF9 and BMP15 reference preparations and cumulus cell extracts, therefore a granulosa cell (GC) extract was used as reference preparation with arbitrary unitage (au) in both ELISAs.

FIG. 20. Linear regression analysis between; (A) cumulus cell total DNA and oocyte number retrieved during an IVF cycle, (B) cumulus cell BMP15 and oocyte number and (C) cumulus cell BMP15 and total DNA. Dots represent individual patients. Note the close relationship between BMP15 and DNA in contrast to BMP15 and oocyte number. This is attributed to varying numbers of cumulus cells per oocyte between patients.

FIG. 21. Relationship between cumulus cell BMP15 levels and oocyte number retrieved during an IVF cycle (dots represent individual patients), when BMP15 is expressed per oocyte (A) and per μg DNA (B). Patients with more oocytes secrete not only more BMP15 in total, but also more BMP15/oocyte (secreted and detected on adjacent cumulus cells).

FIG. 22. Relationship between cumulus cell BMP15 levels (per μg DNA) and patient age. Dots represent individual patients. A significant decline is noted with age in both the regression analysis (A) and when grouped to age <35 and >35 years (B).

FIG. 23. Relationships between; cumulus cell BMP15 levels (per μg DNA) and (A) mature (metaphase II [MIT]) oocyte (%), (B) mature oocyte number; and total cumulus cell BMP15 levels and (C) mature (metaphase II [MIT]) oocyte (%), (D) mature oocyte number. Dots represent individual patients.

FIG. 24. Relationships between; cumulus cell BMP15 levels (per μg DNA) and (A) oocyte fertilisation rate (% 2PN/MII), (B) and number of oocytes successfully fertilised (2PN); and total cumulus cell BMP15 levels and (C) oocyte fertilisation rate (% 2PN/MII), and number of oocytes successfully fertilised (2PN) (D). Dots represent individual patients.

FIG. 25. Relationship between total cumulus cell BMP15 levels in patients undergoing ICSI and their (A) serum progesterone and (B) serum estradiol levels.

### KEY TO THE SEQUENCE LISTING

SEQ ID NO: 1—Amino acid sequence of human GDF9 (UniProtKB/Swiss-Prot Accession No. 060383)

SEQ ID NO: 2—Amino acid sequence of human BMP15 (UniProtKB/Swiss-Prot Accession No. 095972 or Genbank Accession No. NP-005439)

SEQ ID NO: 3—N-terminal peptide of GDF9 (pro-domain)

SEQ ID NO: 4—N-terminal peptide of BMP15 (pro-domain)

SEQ ID NO: 5—C-terminal peptide of GDF9 (mature domain)

SEQ ID NO: 6—C-terminal peptide of BMP15 (mature domain)

SEQ ID NO: 7—N-terminal peptide of GDF9 to which mAb 53-1 is raised

SEQ ID NO: 8—N-terminal peptide of GDF9 to which mAb 72b is raised

SEQ ID NO: 9—N-terminal peptide of BMP15 to which mAb 28A is raised

### DETAILED DESCRIPTION

### General Techniques and Definitions

Unless specifically defined otherwise, all technical and scientific terms used herein shall be taken to have the same meaning as commonly understood by one of ordinary skill in the art (e.g., in immunology, cell biology, protein chemistry and biochemistry).

Unless otherwise indicated, the molecular genetics, biochemistry, and immunological techniques utilized in the present invention are standard procedures, well known to those skilled in the art. Such techniques are described and explained throughout the literature in sources such as, J, Perbal, A Practical Guide to Molecular Cloning, John Wiley and Sons (1984), J. Sambrook and Russell., Molecular Cloning: A Laboratory Manual, 3^{rd} edn, Cold Spring Harbour Laboratory Press (2001), R. Scopes, Protein Purification—Principals and Practice, 3^{rd} edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

BMP15, GDF9 and Cumulin

GDF9, BMP15 and cumulin are unique members of the TGF-β family—GDF9 and BMP15 are essentially only expressed in the gametes (oocytes in females, spermatocytes in males), making them ideal markers of fertility and therapeutic targets. There are possible non-gamete sites of expression of GDF9 and BMP15, however expression levels are substantially lower than in oocytes and spermatocytes and physiological roles for GDF9 and BMP15 have only been found in the gonads.

GDF9 and BMP15 are synthesized as precursor molecules consisting of N-terminal pro- and C-terminal mature domains. During synthesis, the prodomains direct folding and dimerisation of the mature growth factors (Shi, 2011). Furin-like proteases cleave BMP15 and GDF9, which are secreted from oocytes and spermatocytes non-covalently associated with their prodomains. Extracellularly, prodomains may localise mature GDF9 and BMP15 in the vicinity of their target cells. Unlike most TGF-β proteins, GDF9 and BMP15 lack the cysteine residue that forms an intermolecular disulfide bond (Mottershead, 2013) and, therefore, function as non-covalent dimers. Hence, individual monomers of GDF9 and BMP15 are free in principle to assemble into a heterodimer to form cumulin.

It is believed that following prodomain displacement, human BMP15 binds to complexes of type I (ALK6) and type II (BMPRII) receptors on the surface of granulosa cells. Receptor binding leads to the activation of Smad1/5 transcription factors and the expression of genes, such as those involved in cumulus cell expansion (Ptx3, Has2 and Ptgs2). In contrast, human GDF9 remains associated with its prodomain in a latent complex. Even following prodomain removal, mature human GDF9 has very low signaling capacity via Smad2/3.

In mono-ovular species, GDF9 and BMP15 are co-expressed throughout most of oogenesis and, hence, should always be considered in combination (Gilchrist et al., 2008). Indeed, there is evidence for synergistic interactions between GDF9 and BMP15 at genetic, biochemical and functional levels. The present inventors have demonstrated the potent bioactivity of the GDF9:BMP15 heterodimer, cumulin, on ovarian granulosa and cumulus cells relative to GDF9 and BMP15 homodimers (Mottershead et al., 2015). It is evident that this molecule has utility as a fertility diagnostic and in reproductive therapies. Notably, prior to the work of the present inventors, cumulin had not been measured in native biological tissues or fluids.

The major role of GDF9 and BMP15 secreted by oocytes is to regulate the growth and differentiation of its neighbouring granulosa cells (GCs), including cumulus granulosa cells, within the follicle, which in turn supply the oocyte with the support necessary for future healthy embryo/fetal development (Gilchrist et al., 2008). Hence, GDF9, BMP15 and cumulin are paracrine growth factors, with their biological functions ascribed to the immediate microenvironment surrounding oocytes and spermatocytes. They are not thought of as hormones—they have no described role outside the gonads.

A selection of amino acid sequences are provided as examples of GDF9 and BMP15 sequence in SEQ ID NOs: 1 to 6. The skilled person will appreciate that there are other known isoforms, fragments and variants of GDF9 and BMP15, and that the amino acid sequences of these isoforms, fragments and variants can readily be located in well-known sequence databases such as Genbank and UniProtKB/Swiss-Prot.

Detecting and/or Determining the Level of GDF9 and BMP15 in a Sample

The present inventors are the first to describe and comprehensively validate a series of assays for measuring GDF9 and BMP15 in biological samples, particularly serum or plasma. The capacity to detect these growth factors in serum or plasma was unexpected, as GDF9 and BMP15 are paracrine growth factors, principally secreted by oocytes and spermatocytes only, with no known endocrine function. This demonstration of the measurement of oocyte-secreted biomarkers systemically provides for the application of the biomarkers to the diagnosis and treatment of reproductive disease, including infertility.

Any suitable method known to one of skill in the art for detecting the level of biological markers in a patient may be used in the methods and assays described herein for detecting GDF9 and BMP15. Thus, the methods of the invention may involve a degree of quantification to determine levels of biological markers (also referred to as “biomarkers”) in patient samples. Such quantification is readily provided by the inclusion of appropriate control samples or by comparison to reference data.

Compounds that bind a biological marker when used according to the methods described herein may be linked to a reagent such as a detectable label to allow easy detection of binding events in vitro or in vivo. Suitable labels include radioisotopes, dye markers or other imaging reagents for detection and/or localisation of target molecules. Compounds linked to a detectable label can be used with suitable in vivo imaging technologies such as, for example, radiology, fluoroscopy, nuclear magnetic resonance imaging (MRI), CAT-scanning, positron emission tomography (PET), computerized tomography etc. As used herein, the terms “label” and “detectable label” include molecules, but are not limited to, radioactive isotopes, fluorescers (fluorophores), chemiluminescers, chromophores, enzymes, enzyme substrates, enzyme cofactors, enzyme inhibitors, chromophores, dyes, metal ions, metal sols, ligands (e.g., biotin, avidin, strepavidin or haptens), intercalating dyes and the like. The term “fluorescer” or “fluorophore” refers to a substance or a portion thereof which is capable of exhibiting fluorescence in a detectable range.

In one embodiment, the level of GDF9, BMP15 and/or cumulin polypeptide is determined in a patient sample. For example, the method may comprise contacting a biological sample derived from the patient with a compound capable of binding to a GDF9, BMP15 and/or cumulin polypeptide, and detecting the formation of complex between the compound and the polypeptide. Detecting GDF9, BMP15 and/or cumulin polypeptides includes detecting fragments of the polypeptides, including for example, immunogenic fragments or epitopes of the polypeptides.

Compounds that bind GDF9, BMP15 and/or cumulin that are useful in the methods and assays described herein may be any compound, e.g. a polypeptide, ligand or other molecule, identified as having binding affinity to GDF9, BMP15 and/or cumulin. The binding between a compound and GDF9, BMP15 and/or cumulin may be mediated by covalent or non-covalent interactions or a combination of covalent and non-covalent interactions. When the interaction of the compound and GDF9, BMP15 and/or cumulin produces a non-covalently bound complex, the binding which occurs is typically electrostatic, hydrogen-bonding, or the result of hydrophilic/lipophilic interactions. In one embodiment, the compound that is used to detect or bind to GDF9, BMP15 and/or cumulin is an antibody.

A variety of immunoassay formats may be used to select antibodies specifically immunoreactive with GDF9, BMP15 and/or cumulin. For example, solid-phase ELISA immunoassays are routinely used to select antibodies specifically immunoreactive with a protein or carbohydrate. See Harlow and Lane (1988) Antibodies, a Laboratory Manual, Cold Spring Harbor Publications, New York, for a description of immunoassay formats and conditions that can be used to determine specific immunoreactivity.

As is readily appreciated by those of ordinary skill in the art, the immunological binding reagents encompassed by the term “antibody” extend to all forms of antibodies from all species, and antigen binding fragments thereof, including dimeric, trimeric and multimeric antibodies; bispecific antibodies; chimeric antibodies; human and humanized antibodies; recombinant, engineered, camelid and camelized antibodies, and fragments thereof. The term “antibody” is thus used to refer to any antibody-like molecule that has an antigen binding region, including, for example molecules such as antibody fragments (e.g., Fab′, Fab, F(ab′)₂, single domain antibodies (DABs), Fv, scFv (single chain Fv), linear antibodies, diabodies, camelized antibodies and the like. The techniques for preparing and using various antibody-based constructs and fragments are well-known to those of ordinary skill in the art.

In some embodiments, the antibodies bind specifically to GDF9, BMP15 and/or cumulin. The terms “specifically binds”, “bind specifically”, “specific binding” refer to the ability of an antibody to bind to a target molecular species in preference to binding to other molecular species with which the specific binding agent and target molecular species are admixed.

Protein detection systems contemplated herein include any known assay for detecting proteins in a biological sample isolated from a subject, such as, for example, SDS/PAGE, isoelectric focussing, 2-dimensional gel electrophoresis comprising SDS/PAGE and isoelectric focussing, an immunoassay, flow cytometry e.g. fluorescence-activated cell sorting (FACS), a detection based system using an antibody or non-antibody compound, such as, for example, a small molecule (e.g. a chemical compound, agonist, antagonist, allosteric modulator, competitive inhibitor, or non-competitive inhibitor, of the protein). In accordance with these embodiments, an antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, e.g. a high throughput spectroscopy resonance method (e.g. MALDI-TOF, electrospray MS or nano-electrospray MS), are also contemplated. Another suitable protein detection technique involves the use of Multiple Reaction Monitoring (MRM) in LC-MS (LC/MRM-MS).

Immunoassay formats are also suitable, for example, such as those selected from an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

In another embodiment, a nucleic acid detection technique is used. Any suitable technique that allows for the qualitative and/or quantitative assessment of the level of a polynucleotide expressing GDF9, BMP15 and/or cumulin in a sample as known in the art may be used. The terms “nucleic acid molecule” or “polynucleotide” as used herein refer to an oligonucleotide, polynucleotide or any fragment thereof. Comparison may be made by reference to a standard control, a control level, or reference sample or reference level. For example, levels of a transcribed gene can be determined by Northern blotting, and/or RT-PCR. With the advent of quantitative (real-time) PCR, quantitative analysis of gene expression can be achieved by using appropriate primers for the gene of interest. The nucleic acid may be labelled and hybridised on a gene array, in which case the gene concentration will be directly proportional to the intensity of the radioactive or fluorescent signal generated in the array.

As known in the art, the level of a biological marker such as GDF9, BMP15 or cumulin may be determined according to the detection technique used. Thus, the level of a biological marker may be, for example, a level of expression, transcription or translation of a polynucleotide, the level of expression of a polypeptide and/or the concentration of a biological marker in a sample. By way of non-limiting example, the level of a biomarker may be determined or inferred by detection of a label, via colorimetric change, alterations in signal intensities, such as by determining the wavelength or strength of a fluorescent signal, by measuring absorbance or optical density, by measuring radioactive signals. In one embodiment, the level of a biomarker is presented as the concentration of the biological marker in a sample obtained from the patient. A concentration of a biological marker may be presented in any suitable unit such as, for example, ng/ml, μg/ml, mg/ml, pg/μl, pg/ml, nmol/L, or μg/l.

Based on the functional interactions known to exist between GDF9 and BMP15 and the close relationship the inventors found between the levels of GDF9 and BMP15 ELISAs in many male and female clinical samples, the assays may reflect a measure of GDF9:BMP15 heterodimer (i.e. cumulin) or complex and that the clinical changes observed by both ELISAs (GDF9 and BMP15) are a measure of the changing levels of cumulin rather than free GDF9 or BMP15. The skilled person will appreciate, however, that detection of subunits of GDF9 or BMP15 either as homodimers, or as heterodimeric cumulin, is useful for the methods and assays described herein, and will serve as indicators of oocyte and sperm quality, as well as predictors of fertility potential and/or pregnancy success. In light of the present specification, the skilled person will be able to produce assays that discriminate cumulin from GDF9 and BMP15, for example using the cumulin ELISA as described herein which utilized a capture mAb directed to GDF9 and a tracer mAb directed to BMP15.

Predicting Oocyte and Sperm Quality and/or Quantity

The inventors have determined that low or non-detectable levels of GDF9, BMP15 and/or cumulin in serum are associated with a low number of oocytes retrieved during IVF treatment. This demonstrates for the first time that GDF9, BMP15 and/or cumulin levels, particularly in serum, are markers of ovarian reproductive reserve, comparable in some respects to that seen with Anti-Mullerian Hormone (AMH). Accordingly, in one embodiment, oocyte quality or quantity in a patient can be predicted by determining the level of GDF9, BMP15 and/or cumulin in a subject sample, wherein a level of GDF9, BMP15 and/or cumulin that is low compared to a reference sample or reference level, or is non-detectable, is indicative of low oocyte quality and/or quantity.

Further, serum GDF9 was shown to correlate strongly with oocyte number retrieved in non-PCOS patients, this relationship was not evident in PCO(S) patients, indicating altered involvement of these oocyte growth factors in the PCO(S) pathology. Furthermore, serum GDF9 provides additional utility to the existing and commonly used serum AMH test, as the ratio of GDF9:AMH is suppressed in PCO(S) relative to non-PCO(S). The present inventors have also demonstrated, for the first time, that low or negligible levels of GDF9, BMP15 and cumulin are indicative of endometriosis.

In addition, serum GDF9, BMP15 and cumulin levels have not previously been described in males. The low levels of serum GDF9 found in men with poor semen analyses indicates that these blood-based diagnostics could have considerable application in the diagnosis and treatment management of male-factor infertility and other male reproductive diseases. This test would be the first serum test using a marker of sperm quality and provide additional information in fertility treatment. Males with lower levels of GDF9, BMP15 and/or cumulin may be advised to consider a semen analysis. For example, a couple with female-factor infertility where the male had not intended to have a semen analysis performed. Further, for males where these combined tests are suggestive of poor quality semen, the patient may be advised to consider additional treatments. For example, patients treated for infertility by ovulation induction may change their treatment to intra-uterine insemination (IUI) or IVF or intra-cytoplasmic insemination (ICSI), which involve sperm preparation steps. In addition, sperm taken for IUI/IVF may need to be supplemented with growth factors such as GDF9, BMP15 and/or cumulin to increase fertilisation efficiency.

Accordingly, there are provided methods and assays for predicting or determining sperm quality. As used herein, and as would be understood by the person skilled in the art, “sperm quality” refers to sperm quantity, e.g. number of sperm per ml of ejaculate, sperm morphology (i.e. shape) and/or sperm motility (i.e. ability to swim forward). “Sperm abnormality” as used herein refers to an alteration in sperm morphology and/or decrease in sperm motility in comparison to sperm with normal morphology and/or motility.

Predicting Fertility Potential and/or Pregnancy Success

The assays and methods described herein are useful for predicting pregnancy success and directing treatment in assisted reproductive technology (ART) and fertility treatments. As used herein, assisted reproductive technology or ART is a general term referring to methods used to achieve pregnancy by artificial or partially artificial means. Such methods include, but are not limited to, in vitro fertilization (IVF), intracytoplasmic sperm injection (ICSI), cryopreservation, intrauterine insemination (IUI), In Vitro Maturation (IVM), and frozen embryo transfer (FET).

The assay described herein is the first serum test using a marker produced essentially only by the oocyte or sperm to provide additional information in fertility treatment. The following aspects of patient management/treatment would potentially be modified by analysing serum levels of GDF9, BMP15 and/or cumulin:

i) Altered family planning/treatment advice: (e.g.) low marker levels in women or men may influence the patients' urgency in planning for a family, or the patients' decisions to use IVF for fertility treatment, fertility preservation, or social egg/sperm freezing;

ii) Altered patient hormonal stimulation regimes with gonadotropins, e.g. female patients with low levels might be stimulated with higher or longer duration of gonadotropins or with differential stimulation regimes, or conversely patients with high levels of marker might receive milder gonadotropin stimulation to avoid adverse outcomes such as ovarian hyperstimulation syndrome;

iii) Altered patient management according to marker levels if the patient has an existing reproductive condition, such as PCO/PCOS;

iv) Referral for additional diagnostic examination (e.g. full semen analysis; additional blood tests for male-factor infertility) for men with aberrant growth factor levels;

v) Altered laboratory procedures used for oocyte insemination (e.g. use of intra-cytoplasmic insemination (ICSI) instead of IVF for men with aberrant growth factor levels;

vi) Increased likelihood of performing additional investigations (e.g. ultrasound) and treatments (e.g. laparoscopic surgery) for endometriosis to omit or treat this as the cause of infertility.

The terms “subject” and “patient” as used herein refer to a mammal being assessed for treatment and/or being treated. In an embodiment, the mammal is a human, such as a female human. The terms “subject” and “patient” thus encompass individuals in need of assessment of fertility potential, including those who have undergone or are candidates for a fertility treatment such as in vitro fertilization.

As used herein, the term “diagnosis”, and variants thereof such as, but not limited to, “diagnose”, “diagnosed” or “diagnosing” includes any primary diagnosis of a clinical state or diagnosis of recurrent disease or disorder, for example a reproductive disease or disorder.

“Prognosis”, “prognosing” and variants thereof as used herein refer to the likely outcome or course of a disease.

As used herein, the phrase “prediction of therapeutic outcome” and the terms “predicting”, “predictive” and variants thereof refer to determining the probability of response to a therapeutic treatment, for example, determining the likelihood of pregnancy success resulting from in vitro fertilization treatment.

“Reproductive disease” or “reproductive disorder” refers to the diseases, disorders and conditions that affect the functioning of the male and female reproductive systems, such as those associated with reduced fertility in both men and women and which may contribute to problems with fertility, pregnancy, and other aspects of reproduction. Reproductive conditions include, but are not limited to, ovarian reserve, ovarian function, oocyte quality, oocyte quantity, premature ovarian failure, ovarian insufficiency, sperm quality and sperm morphology. In one embodiment, the reproductive disease or disorder is selected from infertility and endometriosis.

The diagnostic, prognostic and predictive methods of the present invention may involve a degree of quantification to determine levels of a compound that binds GDF9, BMP15 and/or cumulin in patient samples. Such quantification is readily provided by the inclusion of appropriate reference samples.

Assays, Kits and Devices

Further provided are devices, such as predictive or diagnostic devices, kits and assays for determining the level of GDF9, BMP15 and/or cumulin in a patient or patient sample. Diagnostic/predictive kits based on the biological markers described above can be developed for use in predicting an individual's response to fertility treatment such as IVF treatment. Such test kits can include devices and instructions that a subject can use to obtain a sample, e.g., blood, plasma, serum or urine in some instances with the aid of a health care provider.

Thus, it will be appreciated that the assays, kits and devices described herein may be used as a “companion diagnostic” to a therapeutic treatment, for example fertility treatment, or method in order to validate or direct the use of the therapeutic. Companion diagnostics are increasingly finding utility in the justification of expensive treatments which only confer benefit to a subset of the population. A companion diagnostic test refers to an in vitro diagnostic device or kit, or an imaging tool, the use of which indicates an increased likelihood of a patient responding to treatment. In-vitro Companion Diagnostic tests measure the expression or presence of a specific biomarker that is linked to a disease condition or therapy.

In one embodiment, the device, for example a diagnostic device, comprises an array. The term “array” or “microarray”, as used herein refers to an ordered arrangement of hybridizable array elements, such as polynucleotide probes (e.g., oligonucleotides), or binding reagents (e.g., antibodies), on a substrate. The substrate can be a solid substrate, such as a glass or silica slide, a bead, a fiber optic binder, or a semi-solid substrate, such as a nitrocellulose membrane. The nucleotide sequences can be DNA, RNA, or any permutations thereof.

In some embodiments, there are provided compositions and kits comprising primers and primer pairs, which allow the specific amplification of biomarker polynucleotides, and probes that selectively or specifically hybridize to biomarker polynucleotides. Probes may be labeled with a detectable marker, such as, for example, a radioisotope, fluorescent compound, bioluminescent compound, a chemiluminescent compound, metal chelator or enzyme. Such probes and primers can be used to detect the presence of polynucleotides in a sample and as a means for detecting cell expressing proteins encoded by the polynucleotides. As will be understood by the skilled artisan, a great many different primers and probes may be prepared based on the sequences provided herein and used effectively to amplify, clone and/or determine the presence and/or levels of the biological markers described herein.

In some embodiments, the device or kit comprises reagents for detecting the presence of GDF9, BMP15 and/or cumulin polypeptides. Such reagents may be antibodies or other binding molecules that specifically bind to a GDF9, BMP15 and/or cumulin polypeptide. The antibodies or binding molecules may be labeled with a detectable marker, such as, for example, a radioisotope, a fluorescent compound, a bioluminescent compound, a chemiluminescent compound, a metal chelator, an enzyme, or a particle. Other reagents for performing binding assays, such as ELISA, may be included in the kit.

The kits may further comprise a carrier being compartmentalized to receive in close confinement one or more containers such as vials, tubes, and the like, each of the containers comprising one of the separate elements to be used in the method. For example, one of the containers may comprise a probe that is or can be detectably labeled. Such probe may be a polynucleotide or antibody specific for a biomarker. Where the kit utilizes nucleic acid hybridization to detect the target nucleic acid, the kit may also have containers containing nucleotide(s) for amplification of the target nucleic acid sequence and/or a container comprising a reporter, such as a biotin-binding protein, such as avidin or streptavidin, bound to a reporter molecule, such as an enzymatic, florescent, or radioisotope label. In one embodiment, one of the containers may comprise an antibody that is or can be detectably labelled and which binds GDF9, BMP15 and/or cumulin as described herein.

The kit of the invention may comprise the container described above and one or more other containers comprising materials desirable from a commercial and user standpoint, including buffers, diluents, filters, needles, syringes, and package inserts with instructions for use. A label may be present on the container to indicate that the composition is used for a specific purpose, and may also indicate directions for use, such as those described above. The kit can further comprise a set of instructions and materials for preparing a tissue or cell sample, for example, blood, plasma or serum, and preparing nucleic acids and/or polypeptides from the sample.

Also disclosed herein are point-of-care devices for use in the disclosed methods. In some embodiments, the disclosed methods may be carried out using a point-of-care device, such as a lateral flow device (for example a lateral flow test strip) that may allow quantification of two or more proteins of interest. Lateral flow devices are available in numerous different configurations, but in one example, a test strip may include a flow path from an upstream sample application area to a test site, such as from a sample application area through a mobilization zone to a capture zone. In various embodiments, the mobilization zone may contain a mobilizable marker that may interact with the protein of interest, and the capture zone may contain a reagent that binds the protein of interest for detection and/or quantification. In other embodiments, exemplary point-of-care devices may include an absorbent medium, such as filter paper, that may include indicia for the placement of the biological sample on the medium.

Samples

A “sample” or “biological sample” encompasses a variety of sample types obtained from a subject or patient. The definition encompasses blood, blood fractions such as serum and plasma, and other liquid samples of biological origin such as saliva, urine or semen, solid tissue samples such as a biopsy specimen or tissue cultures. The sample can be used as obtained directly from the source or following at least one step of (partial) purification. The sample can be prepared in any convenient medium which does not interfere with the method of the invention. Typically, the sample comprises cells or tissues and/or is an aqueous solution or biological fluid comprising cells or tissue. Pre-treatment may involve, for example, diluting viscous fluids, and the like. Treatment of a sample can involve filtration, distillation, separation, concentration, inactivation of interfering components, and the addition of reagents. The selection and pre-treatment of biological samples prior to testing is well known in the art. In a preferred embodiment, the sample is blood or a fraction thereof such as serum or plasma. The term “sample” encompasses a clinical sample, and also includes tissue obtained by surgical resection, tissue or cells obtained during fertility treatment such as IVF, tissue obtained by biopsy, cells in culture, cell supernatants, cell lysates, tissue samples, blood, plasma, serum, saliva, urine and the like.

Reference Sample or Reference Population

In some embodiments, the skilled person will compare the detected level of GDF9, BMP15 and/or cumulin with the levels of GD9, BMP15 and/or cumulin in a reference sample or reference level. For example, the method may comprise measuring the level of GDF9, BMP15 and/or cumulin in a serum or plasma sample, or in a sample comprising cells or follicular fluid, and comparing the level of GDF9, BMP15 and/or cumulin to a reference sample or reference level of GDF9, BMP15 and/or cumulin.

In one embodiment, the reference sample used is a purified form of GDF9, BMP15 or cumulin which exhibits a similar response profile in the assay compared to the test sample i.e. the reference sample is parallel and behaves in the same does-dependant manner as the test sample. A second requirement is that these preparations are stable with storage often at −20 C to −80 C. Such preparations can be isolated from native biological sources or produced using recombinant DNA technology. These reference preparations can be either the full length or fragments of the native molecule.

In another embodiment, the reference sample is obtained from, or reference level is determined from, a sample obtained from a healthy individual, or population of healthy individuals, known not to have a reproductive disorder or disease.

In another embodiment, the reference sample exhibits a similar experimental profile in the assay as compared to the test sample, i.e. the reference sample is parallel and behaves in the same does-dependant manner as the test sample.

In another embodiment, the reference sample or reference level is determined from levels of GDF9, BMP15 and/or cumulin in serum, plasma, or cells from a healthy individual or population of healthy individuals.

As will be known to those skilled in the art, when internal reference samples are not included in each assay conducted, the reference level may be an established data set.

Established data sets may be selected from, for example:

1. a data set comprising measurements of the level of GDF9, BMP15 and/or cumulin in a normal, healthy individual or population of individuals;

2. a data set comprising measurements of the level of GDF9, BMP15 and/or cumulin in an individual or population of individuals treated for a reproductive disease or disorder;

3. a data set comprising measurements of the level of GDF9, BMP15 and/or cumulin from subjects known to have a reproductive disease or disorder;

4. a data set comprising measurements of the level of GDF9, BMP15 and/or cumulin from a subject being tested wherein said measurements have been made previously, such as, for example, when the subject was known to be healthy or, in the case of a subject having a reproductive disease or disorder, when the subject was diagnosed or at an earlier stage in disease progression;

5. a data set comprising measurements of the level of a compound that binds GDF9, BMP15 and/or cumulin in a cell or population of cells for a healthy individual or a population of healthy individuals; and

6. a data set comprising measurements of the level of GDF9, BMP15 and/or cumulin for a normal individual or a population of normal individuals.

In the present context, subjects known to have a reproductive disease or disorder shall be taken to refer to a population or sample of subjects diagnosed with a reproductive disease or disorder that is representative of the spectrum of the patients suffering the condition. This is not to be taken as requiring a strict normal distribution of morphological or clinicopathological parameters in the population, since some variation in such a distribution is permissible. Preferably, a population will exhibit a spectrum of the condition at different stages of disease progression.

In another embodiment, and as will be known to those skilled in the art, data obtained from a sufficiently large sample of the population will normalize, allowing the generation of a data set for determining the average level of GDF9, BMP15 and/or cumulin in a sample or population. Those skilled in the art are readily capable of determining the baseline for comparison in any diagnostic, prognostic or predictive assay as described herein without undue experimentation, based upon the teaching provided herein.

### EXAMPLES

### Example 1. Clinical Samples and Study Cohort

Serum and follicular fluid (hFF) were collected from IVF Australia (IVFA), Sydney Children's Hospital or the Royal Hospital for Women (RHW) and divided into the following clinical groups.

Antagonist Ovarian Stimulation Treatment Cycles:

Women aged 26-45 with male and/or female factor infertility treated at IVFA undergoing an antagonist ovarian stimulation cycle using FSH (Gonal F or Puregon), and with oocyte retrieval.

Exclusion Criteria:

>45 years old, endometriosis, PCO/PCOS, previous/current ovarian hyperstimulation syndrome (OHSS), recurrent miscarriage, poor responsiveness to gonadotropins, repeated implantation failure, currently on any medication (other than Gonal F or Puregon), or other identified ovarian, uterine or endocrine disorders (e.g. fibroids or hypothyroidism).

The included women were seeking treatment primarily due to male factor infertility and female tubal disorders. Blood samples collected before (on day 2 or 3 of cycle) and after daily FSH treatment for 8-13 days (blood collected on approximately days 6, 8, 10, 12). Serum was collected stored for up to two weeks at 4° C. and then frozen at −80° C.

Polycystic Ovarian Disease (PCO)/Polycystic Ovarian Disease Syndrome (PCOS):

Women aged 27-42 with PCO treated at IVFA for infertility, undergoing an antagonist ovarian stimulation cycle using FSH (Gonal F or Puregon), and with oocyte retrieval.

Exclusion Criteria:

>42 yo, not PCO, not on an antagonist stimulation cycle with Gonal F or Puregon, no oocyte retrieval. Blood samples collected as for Antagonist study above.

Natural Cycle Monitoring Cohort: 8 women aged 27-42 years treated at IVFA and having gonadotropin levels monitored across a cycle without ovarian hyperstimulation (i.e. without FSH and without oocyte retrieval). Exclusion criteria: >42 yo, on a hyperstimulation cycle. Serum was collected, stored for up to two weeks at 4° C. and then frozen at −80° C.

Female Age Cohort:

212 females aged 3 to 52 years were randomly selected to assess effect of age on serum ovarian biomarker levels. This included serum samples from 40 young women attending the Sydney Children's Hospital or Royal Hospital for Women for conditions not associated with cancer or fertility (5 pediatric, 18 aged 21-30 years, and 17 aged 31-40 years), as well as 172 women aged 25-52 years (37±4.8; Mean±SD) treated at IVFA for male and female factor infertility. No exclusion criteria. Serum was collected, stored for up to two weeks at 4° C. and then frozen at −80° C.

For further analyses, clinical data were collected from a randomly selected subset of these IVF patients, and combined with the case-control Study 1 data.

Male Cohort:

Serum from 15 males aged 22-56 years treated at IVFA who had a blood sample collected and a semen analysis performed were included. No exclusion criteria. Serum was collected, stored for up to two weeks at 4° C. and then frozen at −80° C.

### Example 2. Cumulus Cell (CC) Preparations from Human Denuded Oocytes

CCs were obtained as a by-product of the intra-cytoplasmic sperm injection (ICSI) procedure following denuding of oocytes prior to fertilisation. The media containing the CCs following removal of the denuded oocyte were frozen on collection, then thawed and CCs recovered following centrifugation. The CCs were extracted with 50 mM phosphate buffer pH7.5 containing 1.5M NaCl, 1 mM phenylmethylsulfonide fluoride (PMSF) in a volume of 500 μl, the cell debris removed by centrifugation and the supernatant assayed as outlined in the GDF9 and BMP15 ELISA procedures. Initial experiments showed that extraction with buffer containing 0.154M or 1M NaCl were ineffective-partially effective while 1.5M and 2M gave maximal extraction. 1.5M NaCl was used in the extraction buffer. The final salt concentration in the ELISA was 1M NaCl.

### Example 3. Samples for GDF9, BMP15 and Cumulin Assay Validation

Blood was collected into serum-separator tubes (SST), and EDTA and heparin-coated tubes from 11 random female blood donors of the RHW. The sera and plasma were processed immediately, aliquoted and stored at −80° C. No exclusion criteria applied. Recombinant preparations of GDF9, BMP15 and cumulin and pools of serum and hFF were prepared as reference or QC preparations in the ELISA. These pools were aliquoted and stored at −80° C. Human male serum deficient in GDF9 and BMP15 immunoactivity used as a buffer constituent in the GDF9 ELISA was obtained from excess blood collected from patients with haemochromatosis. These patients were otherwise healthy. The blood was collected into blood bags and allowed to clot at 4° C. The sera were then collected, aliquoted and stored at −80° C. Male sera pools with undetectable GDF9 levels were used in the ELISA and the same batch was employed with all the serum assays presented herein.

### Example 4. Production and Purification of Recombinant GDF9/BMP15/Cumulin

Pro-GDF9/BMP15 forms were produced by transient transfection in HEK293T cells using PEI-MAX. In brief, cells were plated at 11×10⁶ cells per plate on 15 cm plates, and then transfected GDF9 or BMP15 DNA constructs using PEI-MAX (Polysciences) and Opti-MEM media (Life Technologies, according to the manufacturer's protocol). At 4 hours post-transfection, the transfection media was removed, and replaced with fresh OPTI-MEM media. The following day (24 hours post-transfection), the cells were incubated in production media (DMEM:F12 medium containing L-glutamine, 0.02% BSA, and 0.005% heparin) and incubated for a further 72 hours (total 3 days in production media). Pro-GDF9/BMP15 forms were then isolated from conditioned media by IMAC immunoaffinity. Conditioned media (100 ml) was first concentrated (twice) using centricon devices with a 5 kDa molecular weight cut-off (EMD Millipore, Billerica, Mass.) and resuspended in phosphate buffer (10 mM PO4, 0.5 M NaCl, pH 8.0). Concentrated media was applied to a HisPur™ Cobalt-resin (Thermo Fisher Scientific, MA, USA) and incubated overnight at 4° C. Unbound protein was collected, and the resin washed 4× with phosphate buffer. Bound ligands were eluted with 150 mM imidazole in phosphate buffer. Imidazole was removed by buffer exchange on a PD-10 column (GE Healthcare), and PBS (pH 7.4) with 0.1% BSA was applied to the preparations. The recovery and yield of pro-GDF9/BMP15 preparations were determined by Western blot analysis and ELISAs. Mature (17 k) hGDF9 and hBMP15 were purchased from R&D Systems (Minneapolis, Minn. USA).

A list of GDF9 and BMP15 preparations used in this study is presented in Table 1.

TABLE 1
Preparations used in the GDF9, BMP15 and Cumulin ELISAs

| | |
|---|---|
| GDF9(17k, R&D) | Mature 17k (R&D) |
| hmolwtGDF9CS | hmolwt GHDF9 Full length non-cleaved His Tagged |
| | IMAC purified |
| hmolwtBMP15 | hmolwt BMP15 Full length non-cleaved His Tagged |
| | IMAC purified |
| hCumulin | Non-covalent BMP15_His-8 + GDF9_untagged [symbol] |
| | IMAC purified |
| IVF3,IVFB2IVE1 | Serum pools obtained from women undergoing |
| | gonadotropin-stimulation |
| Female | Serum pools obtained from young women |
| serum pool | |
| human | Pool from women undergoing infertility treatment |
| follicular fluid | |
| Male Serum | Male serum pool from men with negligible GDF9 |
| Pool #5 | immunoactivity used to offset matrix effects in |
| | the ELISAs |

[symbol]

indicates data missing or illegible when filed

Human cumulin was produced by transient co-transfection of hGDF9 and hBMP15 DNA constructs (non-covalent BMP15_His-8+GDF9_untagged) and purified on a Cobalt resin as described above for GDF9 and BMP15.

### Example 5. Gel Filtration HPLC of Serum

In some experiments serum samples were chromatographed on two Superdex 200 gel filtration columns HiLoad 16/60 in series, in running buffer of 50 mM phosphate buffer pH 7.5, 0.154 M NaCl/0.1% Tween 20. The column was calibrated with column markers (Dexran Blue Void volume, bovine serum albumin (67 k) and myoglobin (17 k).

### Example 6. GDF9 ELISA

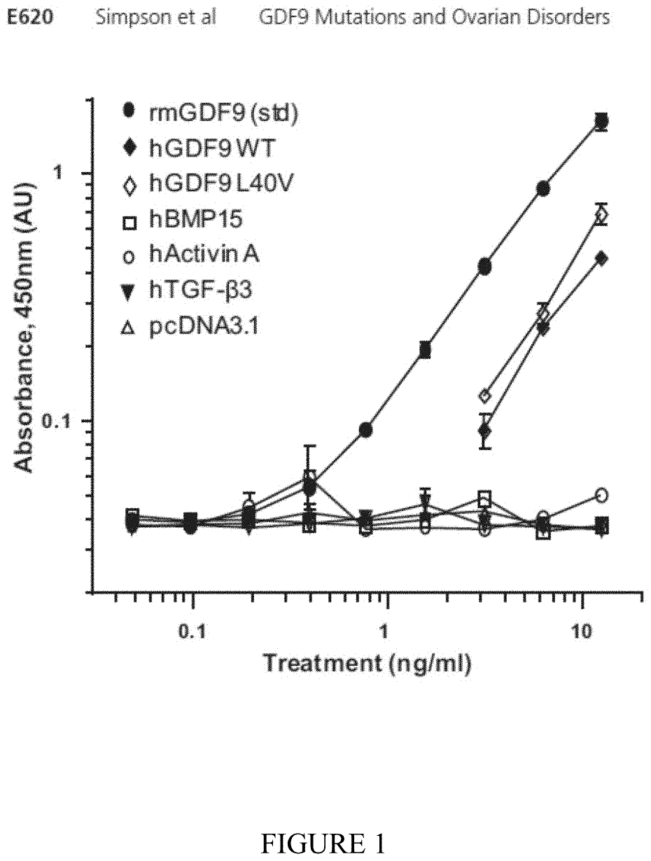

The GDF9 ELISA used is an adaptation of a previously published procedure by our group and collaborators (Simpson et al. 2014) used to quantitate recombinant GDF9 in conditioned media from transfected cell lines producing wild type and mutant human GDF9 (FIG. 1).

The ELISA showed <0.1% cross reaction with mature human BMP15, human activin A and human TGF-β3 (FIG. 1, 2). Both precursor and mature forms of GDF9 were detected in the ELISA. The ELISA consisted of 2 monoclonal antibodies (capture mAb 72B, Oxford Brookes University (OBU), Oxford, UK) and biotinylated mAb 53-1 (OBU) as label. mAb 53-1 is raised to a N-terminal peptide (VPAKYSPLSVLTIEPDGSIAYKEYEDMIATKC (SEQ ID NO: 7)) of the mature region of human GDF9 where the epitope region was localised to a GDF9 specific region EPDG. mAb 53-1 has been shown previously to exhibit strong GDF9 bioneutralising activity (Gilchrist et al. 2004). Western blot analysis of mouse oocyte culture medium showed molecular weight bands of 17.5 k and 57 k consistent with mature and precursor GDF9, respectively. mAb 72b was directed to a N-terminal peptide (KKPLGPASFNLSEYFC (SEQ ID NO: 8)) sequence of GDF9. No further information has been found in the literature about this mAb. Mature form (17 k) of GDF9 (R&D Systems) were used as the reference preparation in this ELISA.

96-well Maxi-sorp plates (Perkin Elmer, Waltham, Mass.) were coated with 72B mAb (500 ng/well in 50 mM Na₂CO₃ pH 9.6) overnight at room temperature, washed and blocked with 300 μl 50 mM Tris/HCl pH7.8, 1% BSA. The plate was washed with wash buffer (12.5 mM Tris/HCl pH 7.5, 0.39M NaCl, 0.125% Tween 20) prior to assay.

Several assay designs were employed:

1. Purified GDF9 preparations or GDF9 preparations in culture medium, sample and standard were added in a total volume of 200 μl in 50 mMTris/HCl pH 7.5 containing 0.154 M NaCl, 0.5% BSA, 0.1% Tween 20.

2. Serum/hFF and GDF9 standard were serially diluted in male serum (devoid of GDF9) for a total well volume of 100 μl serum in a final volume of 200 μl. 2. For serum or human follicular fluid, serum/hFF (225 μl) and buffer (225 μl, 200 mM Tris/HCl pH 8.0 containing 2 M NaCl, 1% BSA, 2% Tween 20, 10-50 μg/ml mouse IgG) were premixed and preincubated for 1 hr at room temperature prior to addition (200 μl in duplicate) to the mAb-coated microtitre plate, followed by an overnight incubation at 4° C. The plate was then washed 6 times with wash buffer. Biotinylated mAb 53-1 (40-60 ng/100 μl in 50 mM Tris/HCl pH 7.5 containing 0.154 M NaCl, 0.5% BSA, 0.1% Tween 20) was added and the plate incubated for 2 h at room temperature. The plate was washed 5× followed by the addition of Streptavidin-HRP (1:3000 SNN 2004 (Invitrogen), 45 min room temperature), washed 6× followed by the addition of tetramethylbenzidine (Sigma-Aldrich, St Louis, Mo.). Reaction was stopped with 1 M H₂SO₄ with absorbance read at 450 nm.

3. Cumulus Cell extracts. The CC extract was serially diluted in extraction buffer (50 mM phosphate buffer pH 7.5 containing 1.5 M NaCl, 1 mM PMSF) prior to assay. The ELISA consisted of sample or standard (100 μl) in extraction buffer and buffer (100 μl, 50 mM phosphate buffer pH 7.5 containing 0.5M NaCl, 0.2% BSA) using the ELISA assay conditions as outlined above except the initial incubation was overnight at room temperature.

### Example 7. BMP15 ELISA

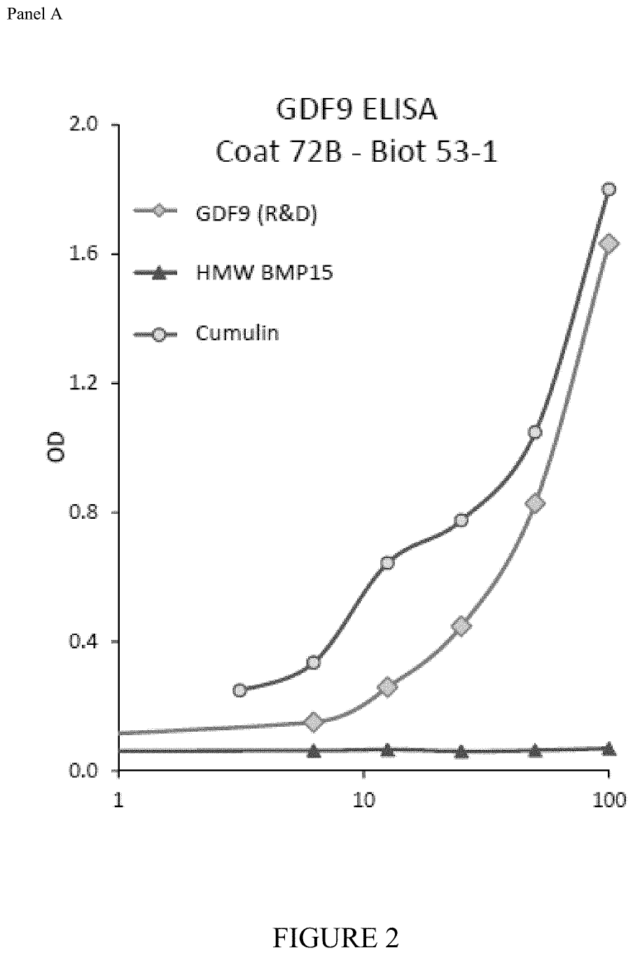

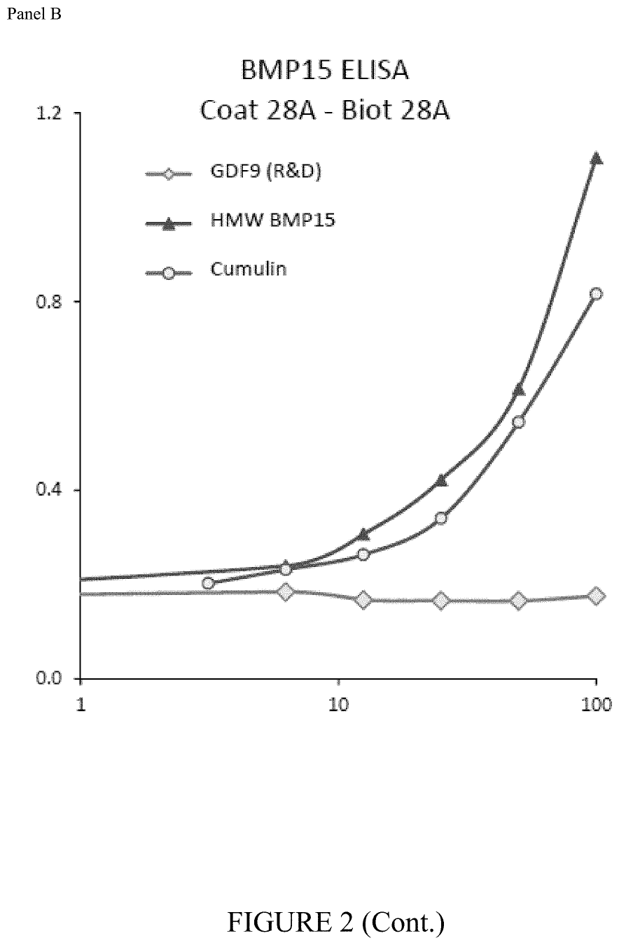

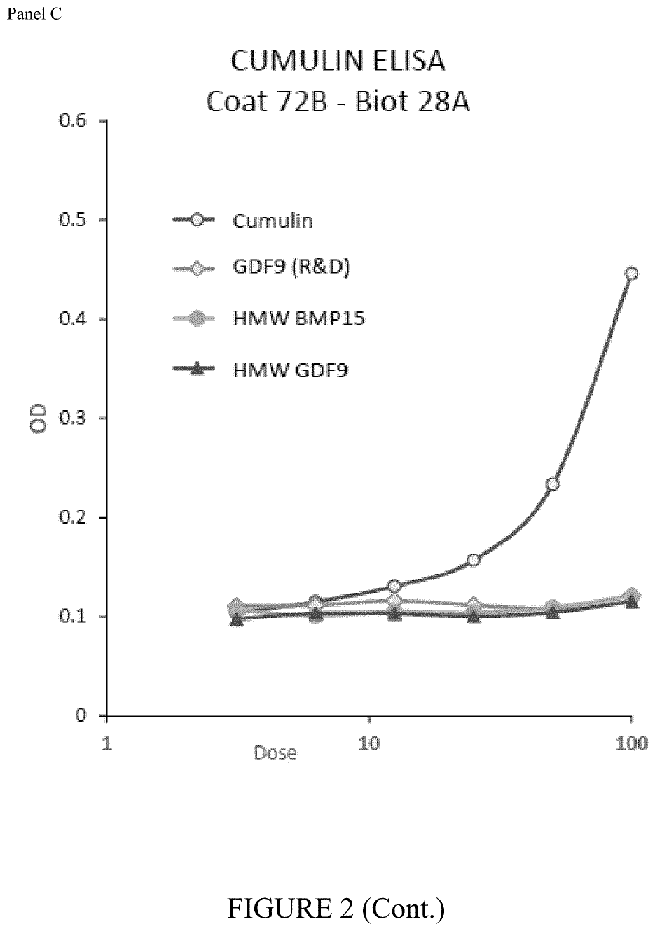

The BMP15 ELISA consists of one antibody (mAb 28A, OBU) as both capture and label (biot-mAb). The 28A mAb is directed to N-terminal peptide of the mature region of hBMP15. 28A (SEVTASSSKHSGPENNQC (SEQ ID NO: 9)). Biotinylation procedure of mAb28A was similar to that reported with GDF9. The antibody reacts strongly with human BMP15 and does not cross react with human GDF9 (FIG. 2). The antibody has been used for immunoblotting of BMP15 and for immunocytochemistry of ovary sections.

The BMP15 ELISA method in application to serum and hFF was modelled closely on the GDF9 ELISA procedure. The preferred assay conditions differed in terms of incubation conditions (initial incubation overnight at room temperature) instead of overnight at 4° C. for the GDF9 ELISA but was otherwise identical. In some early studies the same IVF serum reference preparation was used as standard with same designated unitage. In subsequent studies a purified recombinant hBMP15 preparation was used as standard. The between and within assay variation is presented in Table 2 and showed acceptable between assay and within assay variations with serum samples.

TABLE 2
Validity criteria for GDF9 and BMP15 ELISAs

| | | | | Between | Within | | | Between | Within |
|---|---|---|---|---|---|---|---|---|---|
| | | | | Assay | Assay | | | Assay | Assay |
| IVF serum Pool | No. | GDF9 ELISA | | Variation | Variation | BMP15 ELISA | | Variation | Variation |
| B1 std* | Expts | mean | sd | cv | cv | mean | sd | cv | cv |
| Sensitivity (aU) | 5 | 4.7 | | | | 17.3 | | | |
| IVF3 Serum Pool | 5 | 79.5 | 3.2 | 4.1 | 3.7 | 67.1 | 7.1 | 10.6 | 6.7 |
| QC | | | | | | | | | |
| Sydney hFF QC | 5 | 61.4 | 4.5 | 7.3 | 7.9 | 40.2 | 5.7 | 14.2 | 20.8 |
| IVF E1 Serum Pool | 5 | 17.9 | 2.2 | 12.3 | 13.3 | 44.1 | 8.9 | 20.2 | 27.9 |
| Low QC | | | | | | | | | |
| Female Serum | 5 | 102 | 10.9 | 10.7 | 9.8 | 116 | 8.7 | 7.4 | 11.4 |
| Pool QC | | | | | | | | | |
| | | | Mean | 8.6 | 8.7 | | | 13.1 | 16.7 |

*The unitage of the IVF serum B1 standard is arbitrary and expressed as aU/sample (where 100 μl IVF serum B1 standard = 100 aU)

### Example 8. Cumulin ELISA

Prior to these studies no cumulin ELISA has been described. The mAbs used in the GDF9 and BMP15 ELISA were cross matched to form an ELISA format which would detect GDF9:BMP15 heterodimeric complexes, i.e cumulin. Using similar methodologies to those used with GDF9 and BMP15 ELISAs, an antibody directed to GDF9 (72B) was used as capture antibody in the Cumulin ELISA. The methodology as outlined for GDF9 ELISA above was followed except the detection antibody used (28A) was directed to BMP15. In some assays the serum standard used in the GDF9 and BMP15 ELISAs was also used in the Cumulin ELISA. This Cumulin ELISA shows minimal cross-reaction with GDF9 and BMP15 (FIG. 2C).

TABLE 3
Antibodies used in the respective ELISAs

| | | Biotinylated or |
|---|---|---|
| | Coating antibody | detection antibody |
| GDF9 ELISA | 72B | 53-1 |
| BMP15 ELISA | 28A | 28A |
| Cumulin ELISA | 72B | 28A |

GDF9 specific antibodies : mAb#72B and #53-1
BMP15 specific antibody; mAb#28A

### Example 9. Results

BMP15 ELISA: Application to Serum/Plasma and hFF

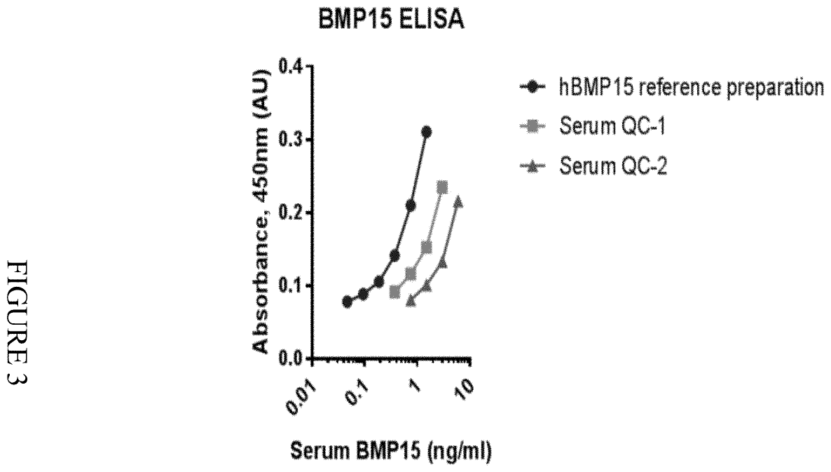

Dose response curves of BMP15 preparations in the BMP15 ELISA are presented in FIG. 3. Parallelism was observed between hmolwt BMP15 as reference preparation and serum dose response curves.

The final assay conditions were defined as those which gave minimal deviations between dose response curves of standard and serum, yet maintained maximal assay sensitivity. Thus the assay consisted of a 1:1 mixture of a) serum or BMP15 std in male serum and b) Tris buffer (200 mM Tris/HCl pH 8.0 containing 2 M NaCl, 0.5% BSA, 0.1% Tween 20, 20-100 ug/ml mouse IgG) with a 1 h pre-incubation prior to addition to the mAb-coated microtitre plate. This was followed by an overnight incubation at room temperature, a 2 hour incubation with biot-mAb 28A and a 45 min Streptavadin-HRP incubation.

Additional experiments were undertaken to assess BMP15 levels in serum, and plasma (using either EDTA or heparin as anticoagulants) following freezing and storage at −80 C. No differences in BMP15 levels were detected between collecting blood in EDTA or serum (109±0.11%) however a decrease (25±3%) in BMP15 levels was observed between heparinised blood and serum.

GDF9 ELISA: Application to Human Serum, hFF and CC Extracts

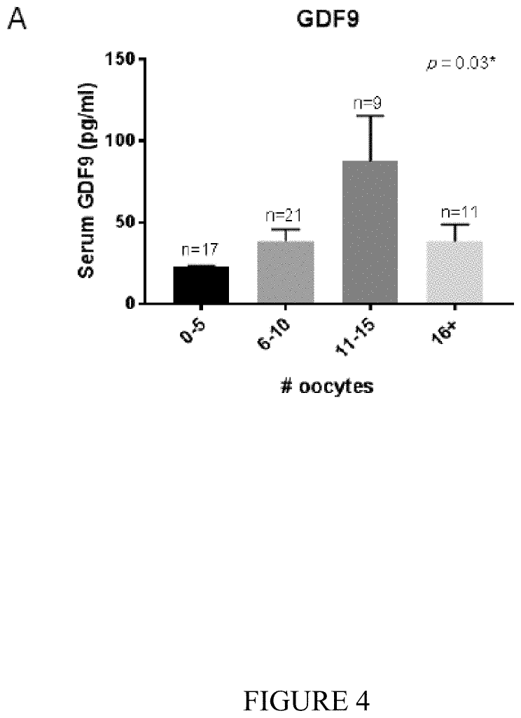

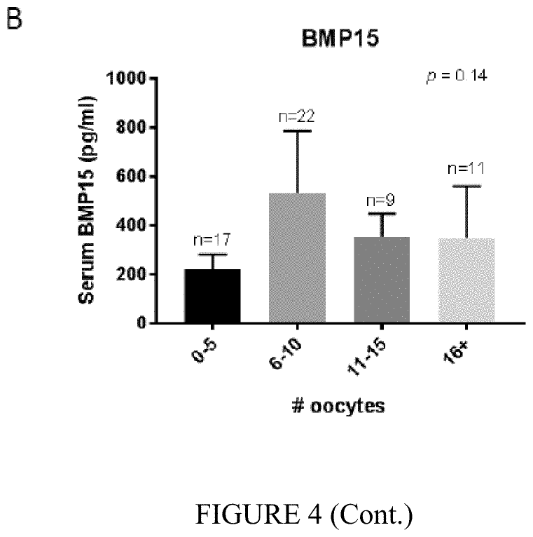

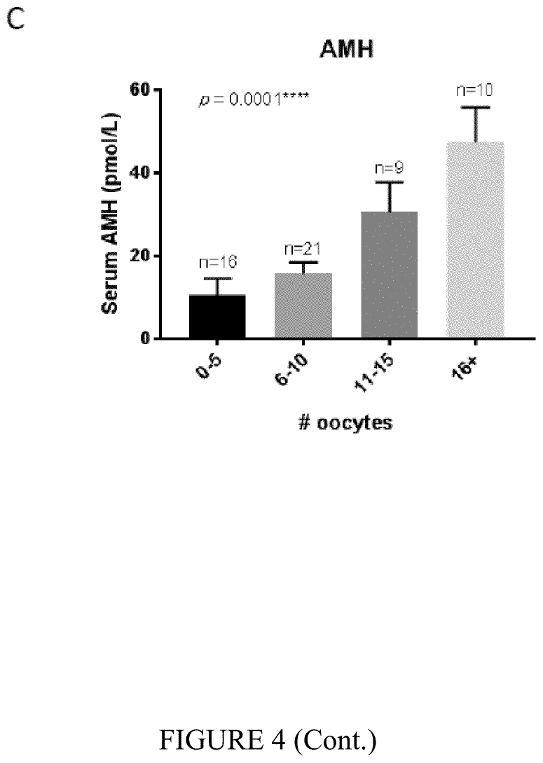

Dose response curves of GDF9 preparations, female serum and follicular fluid in the GDF9 ELISA using final standardised methods are presented in FIG. 2A. Initially non-parallelism was observed between GDF9 reference preparations (17 k GDF9 (R&D) and a precursor GDF9 preparation ˜60 k) and serum/hFF. However, subsequently, parallelism was observed between GDF9 reference preparations (17 k GDF9 (R&D) and a precursor GDF9 preparation ˜60 k). Dose response curves of GDF9 preparations in the GDF9 ELISA are presented in FIG. 4.

A number of initial studies were undertaken to identify or attempt to eliminate the basis for the observed non-parallelism. These studies explored assay conditions for the initial sample incubation, such as incubation time (2-24 h), assay temperature (room temperature (RT) vs 4° C.), Tris buffer concentration (50-100 mM Tris fc), pH (7.5 vs 8.0) and the effects of various detergents (sodium deoxycholate (0.5%), B-D-Octyl glucoside (0.1-1%), sodium dodecyl sulphate (0.1%), Tween 20 (0.1-2%), Triton-X-100 (0.1-2%), RIPA buffer (1% Triton-X-100, 0.1% SDS, 0.5% DOC). The effects of heparin sulphate (−0.6 mg/ml) and protamine sulphate hexamethidine were also examined. Other factors examined were ionic strength (0.15M-2M NaCl, fc), the influence of pre-incubation of serum in the presence of assay buffer before assay and the addition of GDF9-deplete human male serum to standard and serial dilutions of serum/hFF to offset serum matrix effects in the ELISA.

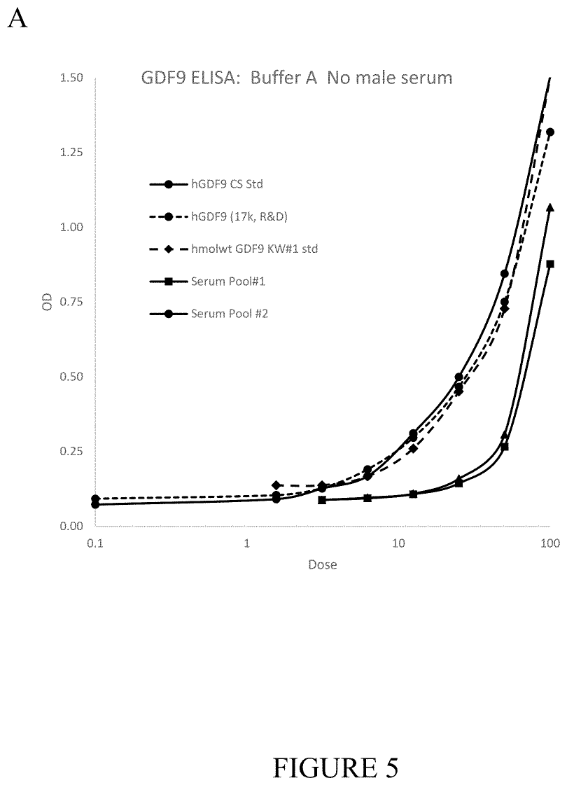

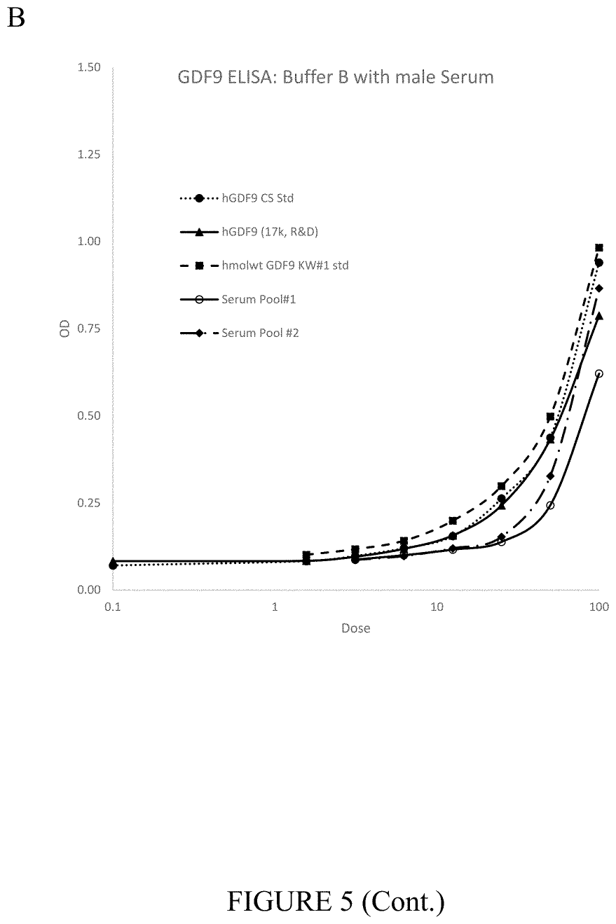

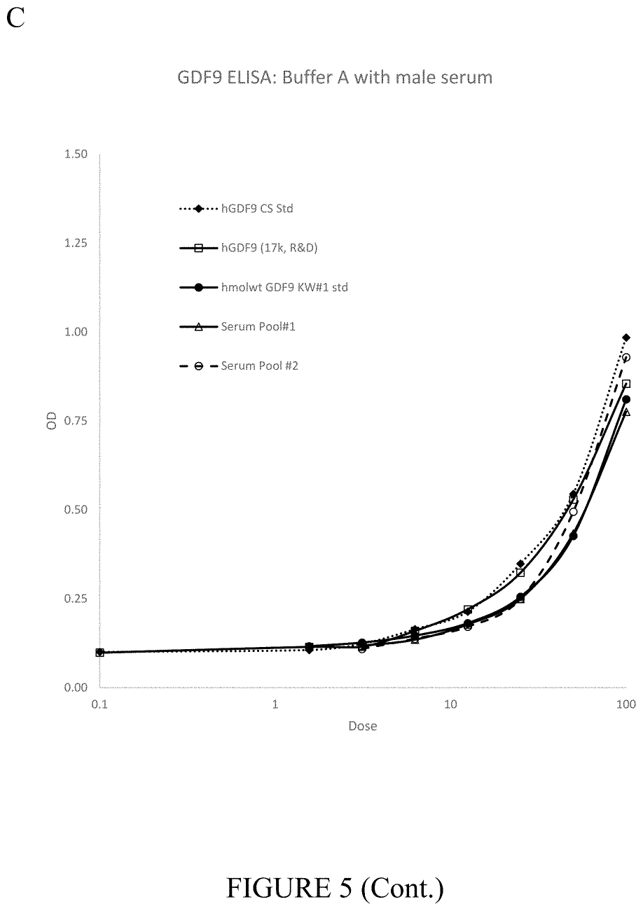

The effects of the addition of male serum with and without the co-addition of 1M NaCl (fc) on the dose response curves of GDF9 standard and serum pool are presented in FIG. 5. The slope of the GDF9 standard remained unchanged while the serum/hFF pools showed a flattening of the dose response curve to approach but not match the slope of the GDF9 standard. The effects of the addition of male serum and 1M NaCl in the ELISA were complex. Both factors had little effect on the shape of the dose response curve of mature GDF9 (FIG. 5A, 5B), while inhibitory effects on hmolwt GDF9 immunoactivity were observed in the presence of male serum. This inhibitory effect was not attributed to the presence of proteoglycans (e.g. heparin sulphate) known to bind GDF9 which showed no interference in the ELISA at concentrations found in serum. The presence of salt is responsible for the flattening of the sera dose response curves. This salt effect is attributed to its interference in GDF9 binding to unknown binding proteins in serum.

The final assay conditions were defined as those which gave minimal deviations between dose response curves of standard and serum/hFF, yet maintained maximal assay sensitivity. Thus the assay consisted of a 1:1 mixture of a) serum or GDF9 std in male serum and b) Tris buffer (200 mM Tris/HCl pH 8.0 containing 2 M NaCl, 0.5% BSA, 0.1% Tween 20) with a 1 h pre-incubation prior to addition to the mAb-coated microtitre plate. This was followed by an overnight incubation at room temperature, a 2 hour incubation with biot-mAb 53 and a 45 min Streptavadin-HRP incubation. A female serum pool with high GDF9 immunoactivity was used as a reference preparation in the measurement of GDF9 in serum/hFF samples. This serum pool was given an arbitrary unitage (aU) of 100 aU/100 ul serum/hFF.

Using these conditions a series of replicate experiments were undertaken to assess the between and within assay variation and general assay reliability of the GDF9 ELISA. These data are presented in FIG. 5 and Table 2. The between assay variation was assessed from the CV of the repeated measurements of female serum and hFF QC pools and gave an average value of 8.6%. The within assay variation was assessed from the CV of measurements at each dilution corrected for dilution within each sample with an average value of 8.7%. These assay criteria assessments indicate that the ELISA was reliable in measuring serum and hFF preparations.

Additional experiments were undertaken to assess GDF9 levels in serum, and plasma (using either EDTA or heparin as anticoagulants). No differences in GDF9 levels were detected between these differing blood collection methods and notable differences in GDF9 levels between individual female subjects were consistent in serum and plasma (Table 4).

TABLE 4
GDF9 levels in matched serum and plasma (EDTA and heparin)
from 13 women

| | KC#58:16 | | | | | |
|---|---|---|---|---|---|---|
| | GDF9 ELISA | | aU/sample | | | |
| GDF9 ELISA | | Plasma | Plasma | | | |
| Female subject | Serum | (EDTA) | (heparin) | Mean | SD | CV |
| 3 | 69.9 | 68.0 | 67.3 | 68.4 | 1.31 | 1.92 |
| 5 | 4.14 | 6.31 | 6.64 | 5.70 | 1.36 | 23.9 |
| 6 | 4380 | 3644 | 3767 | 3930 | 394 | 10.0 |
| 9 | 19.5 | 19.3 | 19.3 | 19.4 | 0.11 | 0.58 |
| 10 | 45.3 | 45.7 | 44.0 | 45.0 | 0.88 | 1.96 |
| 13 | 21.8 | 21.4 | 21.6 | 21.6 | 0.23 | 1.06 |
| Additional 7 | ≤2.5 | ≤2.5 | ≤2.5 | | average | 6.57 |
| subjects | | | | | CV | |

Stability Studies:

The stability of the serum with storage in the GDF9 ELISA was investigated by measuring serum samples after storage for 1 and 2 days at 4° C. and at RT and freeze/thaw of samples either 3 or 6 times. No significant effects of these treatments were observed on GDF9 levels. The average coefficient of variation of OD values between control, 3× freeze/thaw, 6× freeze/thaw, 1 day at RT, 2 days at RT and 1 day at 4° C. and 2 days at 4° C. for serum pools from; a) women undergoing gonadotrophin stimulation, b) asymptomatic young women, c) human follicular fluid, and d) male serum, was 8.2% (range 3.8-11.7%). This indicates that the variation between these various treatments is comparable with the within assay variation indicating minimal effects of sample storage or pre-treatment in the ELISA.

Cumulin ELISA:

A Cumulin ELISA was investigated whereby the capture mAb was directed to GDF9 and the tracer mAb to BMP15 (Table 3; FIG. 2C). Purified preparations of GDF9 and BMP15 at the maximum dose used in their respective GDF9 and BMP15 ELISAs showed no cross reaction in the Cumulin ELISA (FIG. 2C), discriminating this Cumulin ELISA as unique from the GDF9 and BMP15 ELISAs.

Evidence of Cumulin in Human Serum

The identical results obtained by GDF9 and BMP15 ELISAs in application to serum (slope 0.889+/−0.04, correlation coefficient 0.99, p<0.000) strongly suggests that both ELISAs are detecting a related entity in serum, despite the fact that both ELISAs are specific for their respective ligands. A candidate molecule (cumulin) has been hypothesised which is a heterodimer of the GDF9 and BMP15 chains. However evidence of cumulin has not been identified to date in native biological samples.

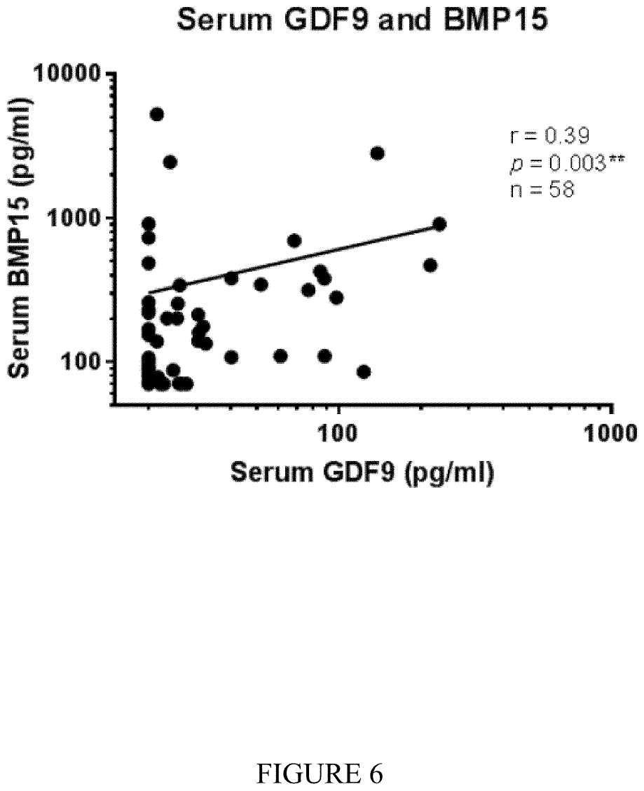

To further test the hypothesis that this immunoactive material is cumulin or cumulin-like, a female serum sample (#6) with very high GDF9 immunoactivity was fractionated by gel filtration (GF-HPLC) and the recovered fractions measured by ELISAs for GDF9, BMP15, Cumulin (FIG. 6). One major peak centred at tube 50 was observed with all ELISAs. This profile based on its elution pattern in comparison with protein standards (e.g. BSA and Myoglobin), corresponds to a molecular weight of 70-90 k with no evidence of smaller molecules consistent with a processed GDF9:BMP15 heterodimer (i.e. cumulin). These data support the hypothesis that GDF9 and BMP15 naturally form a cumulin complex in vivo that can be detected by an ELISA using mAbs specifically detecting both proteins.

Thus, the present inventors are the first to report of the development of a cumulin ELISA, and the first demonstration of native cumulin. This suggests that cumulin or a GDF9:BMP15 complex resembling cumulin, may be the predominant form of GDF9 and BMP15 in human serum and tissues.

Application of GDF9, BMP15 and Cumulin ELISAs to Clinical Samples

The GDF9, BMP15 and Cumulin ELISAs were applied to sera obtained from patients undergoing infertility treatments and compared to endocrine, embryology and clinical parameters. To minimise the effect of any confounders, an initial study included a control cohort of women undergoing IVF on an antagonist ovarian stimulation cycle, excluding women with any significant reproductive abnormalities (‘ANTU’). This was compared to a group of women with polycystic ovaries (with and without the syndrome) also on an antagonist stimulation cycle, AMH, pregnancy, endometriosis, and age. Male sera were analysed relative to semen analysis.

Serum GDF9, BMP15 and Cumulin Levels Correlate with the Number of Eggs Retrieved During IVF

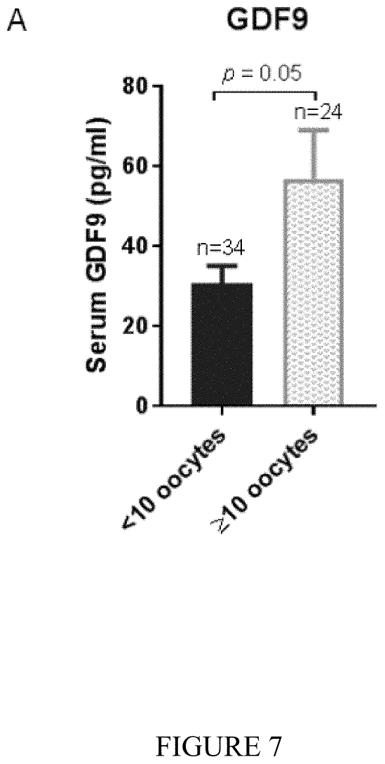

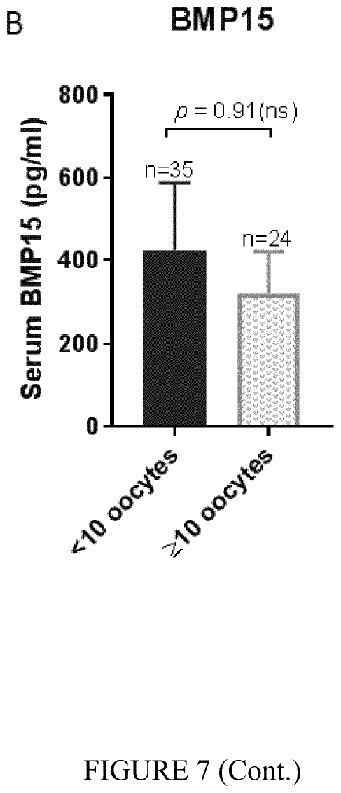

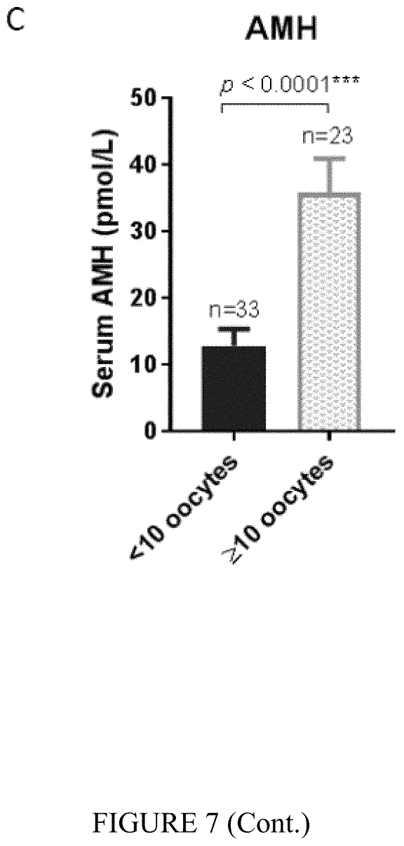

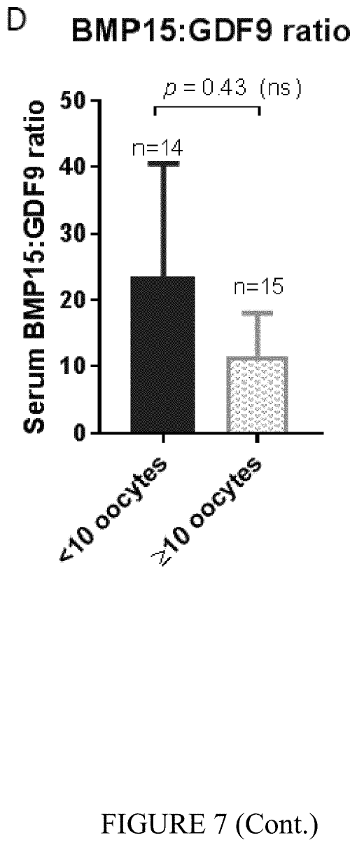

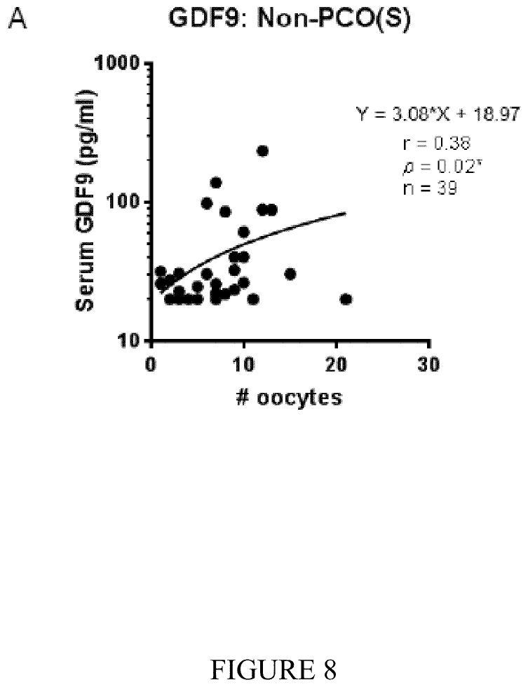

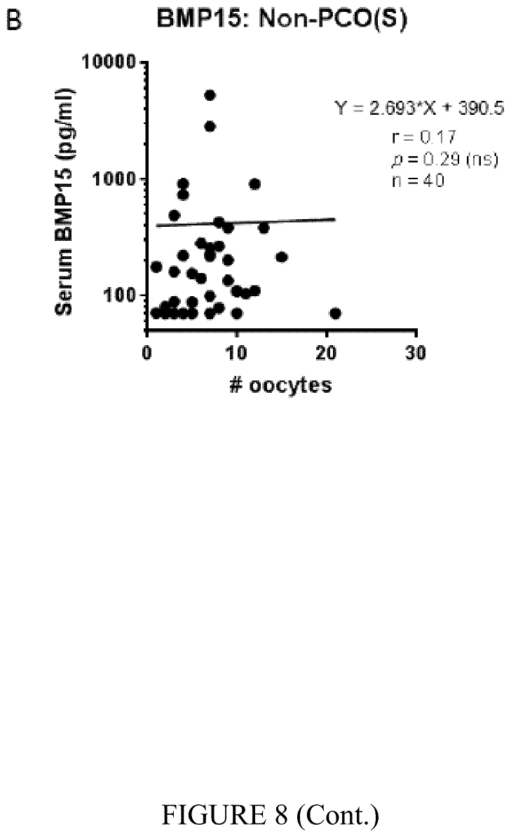

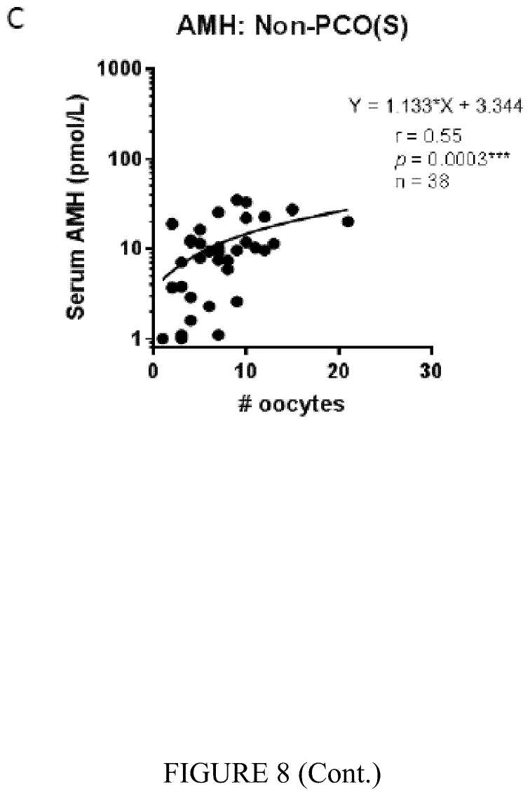

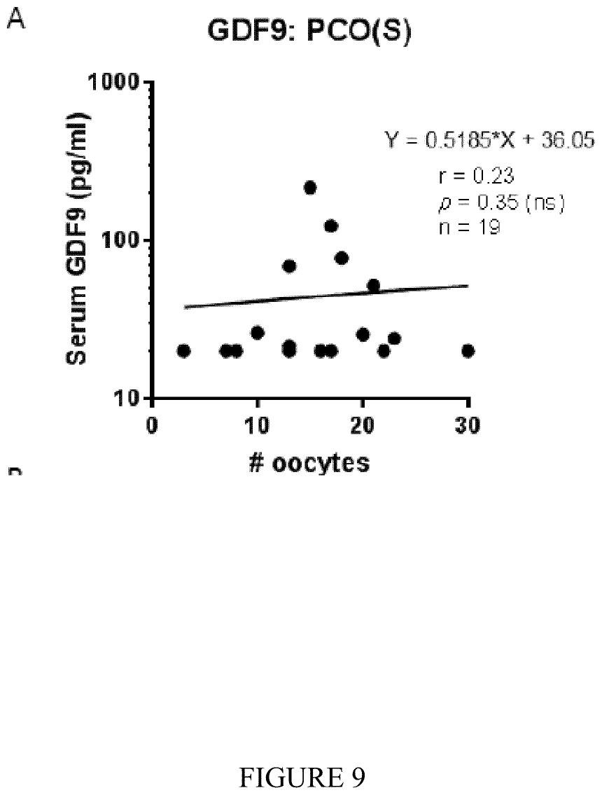

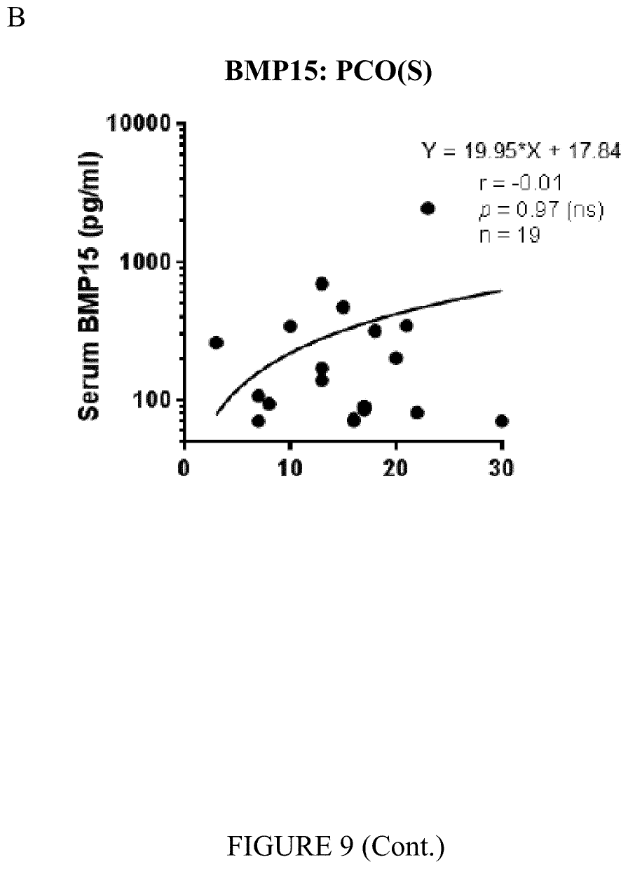

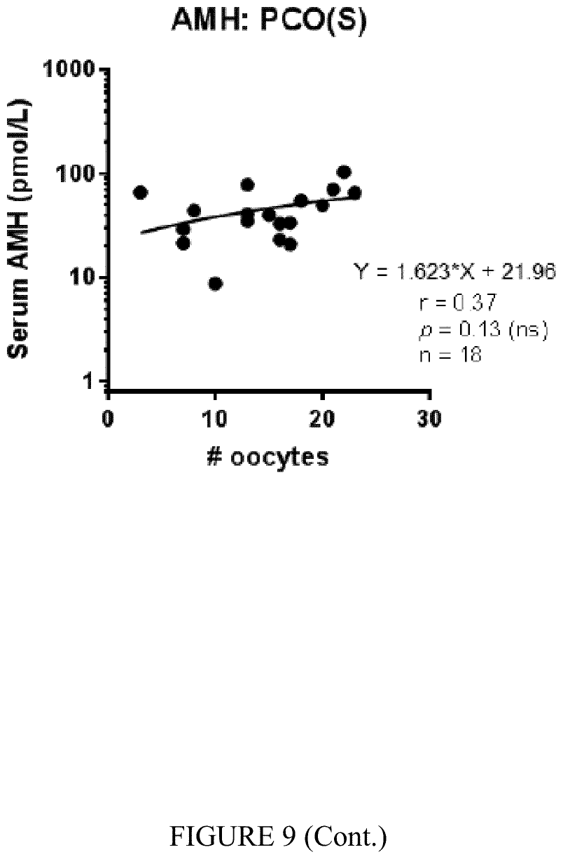

Serum GDF9 levels in the group without PCO(S) (ANTG group) demonstrated a significant trend towards increasing GDF9 with increased number of oocytes retrieved at collection following an ovarian stimulation cycle (FIG. 4A, 7A). A trend was also evident for BMP15 (FIG. 4B) and AMH (FIG. 9C). As expected AMH showed a significant relationship with oocyte number (FIGS. 4C, 7C). As seen in FIG. 6, serum GDF9 correlates significantly (p=0.003) with BMP15.

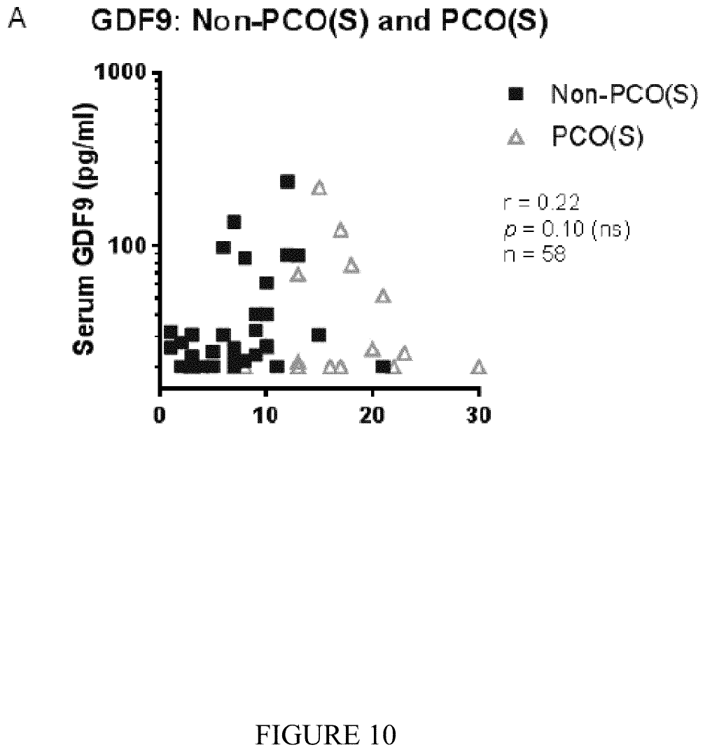

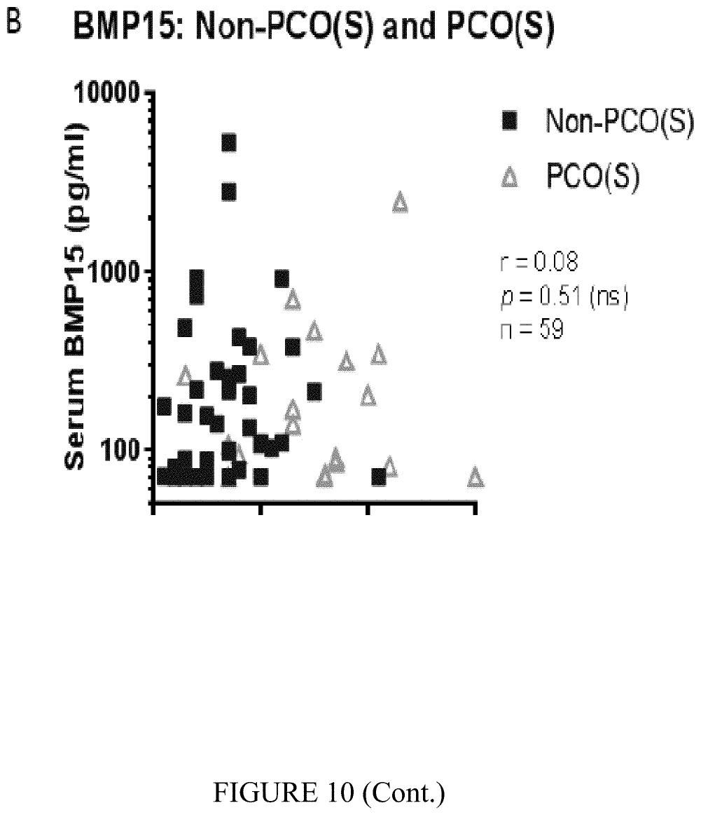

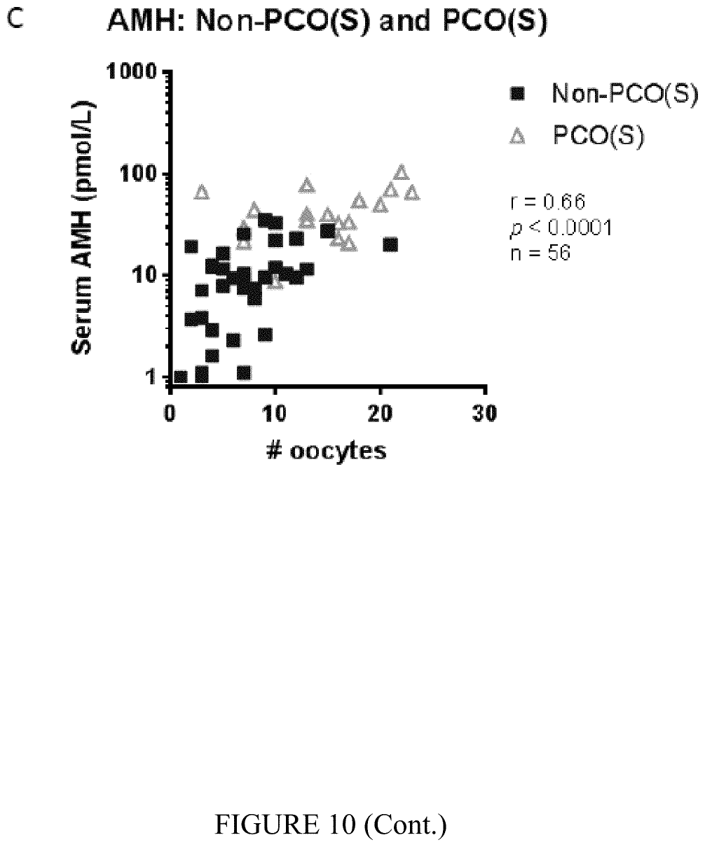

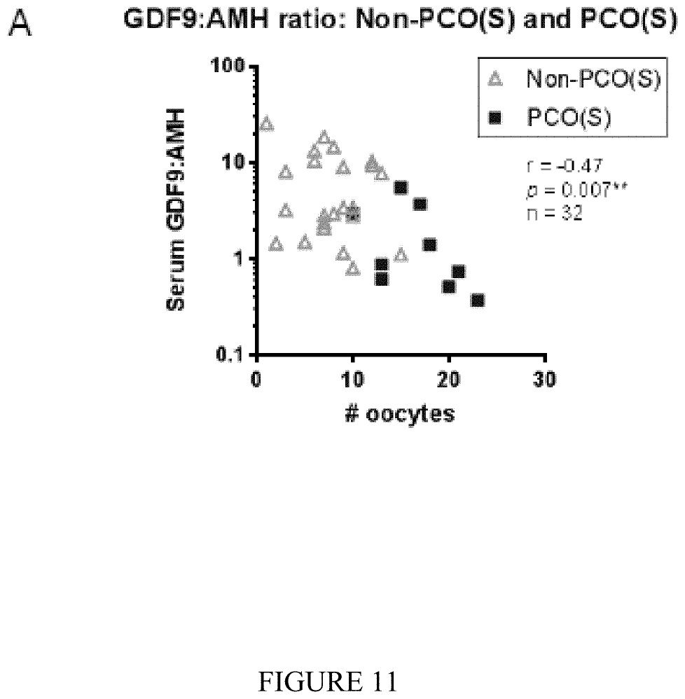

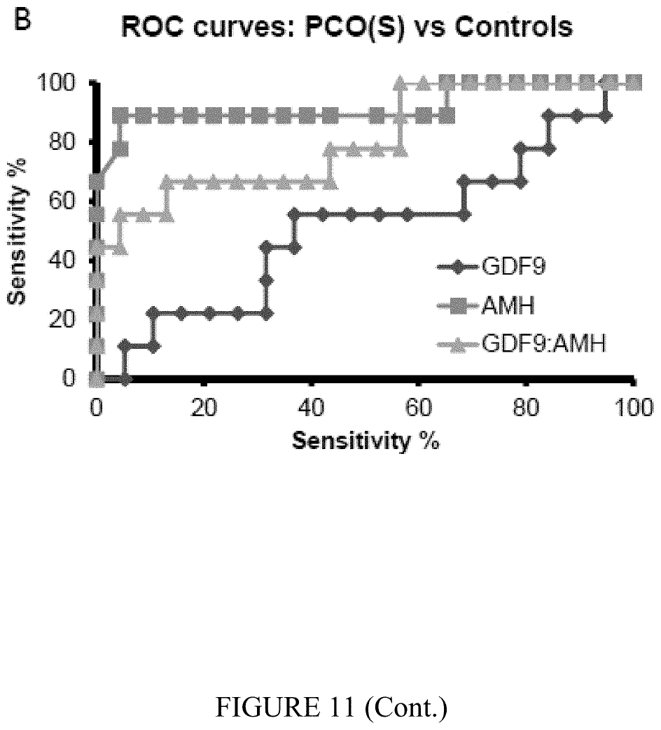

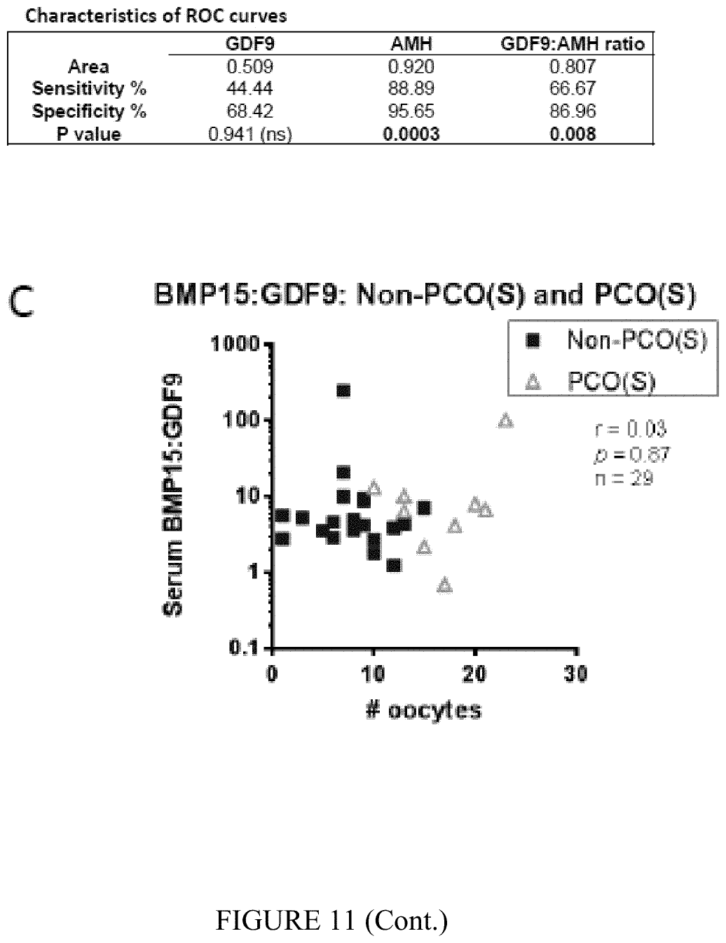

The trends observed in all patient samples were also seen in the group without PCO(S) (FIGS. 7, 10) while in PCO(S) patients, there was no relationship between number of oocytes retrieved and serum GDF9 (FIG. 9A, 10A), BMP15 (FIG. 9B, 10B), or AMH levels (FIG. 9C).

Serum GDF9, BMP15 and Cumulin Correlate Closely with Each Other:

We Found that serum GDF9 correlates with BMP15 and with Cumulin to a very high degree with correlation coefficients >0.95 and slopes of the regression lines ranging between 1.08 and 1.4. These data were generated with the same serum standard in all ELISAs. The intercept of the regression line is also close to the origin. These data indicate that the three ELISA are showing very similar immunological responses with all serum samples. A second Cumulin ELISA was also developed using a different combination of mAbs (capture mAb 28A, tracer mAb 53-1) and obtained very similar results to the other ELISAs supporting the observed relationships.

The serum GDF9 data obtained was extended with the addition of data from an additional 20 patients for which data were available on oocyte number and PCO(S) diagnosis. Comparison of the GDF9 level with number of oocytes retrieved for all patients (n=43), demonstrated a significant increase in serum GDF9 with increased number of eggs retrieved (p<0.05. As expected this correlation was also highly significant for AMH (p<0.0001).

When analysed relative to PCO(S) diagnosis, a significant correlation was observed in non-PCO(S) patients for GDF9 and egg numbers (p<0.05), and for AMH and egg numbers (p<0.001). However, for PCO(S) patients, both these associations were not evident. When further stratified relative to number of oocytes retrieved (<10 and >10), this correlation was also demonstrated for non-PCO(S) patients only, for GDF9 and AMH (p<0.01 and p<0.05, respectively).

Therefore, similar to AMH, increasing serum GDF9, BMP15 and Cumulin levels correlate with increasing number of oocytes retrieved in an IVF ovarian stimulation cycle, particularly for patients without PCO(S).

Using Serum GDF9 and BMP15 Levels in Combination with AMH for Diagnosing PCO(S)

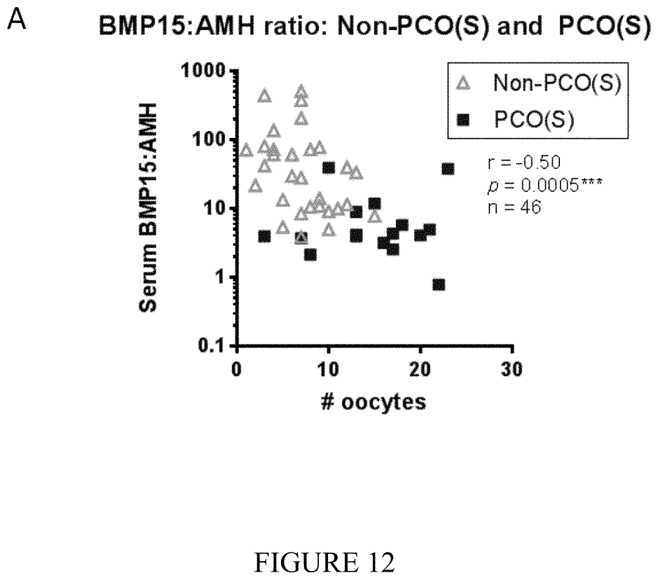

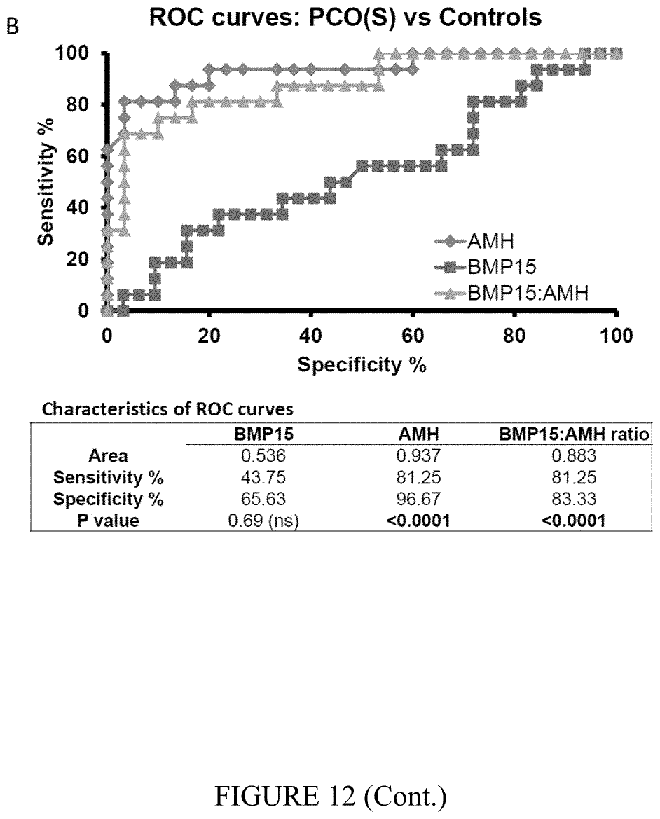

Based on the association between AMH levels and GDF9, a ROC curve analysis was undertaken to assess whether the combined use of GDF9 and AMH or BMP15 and AMH could be used as diagnostic tests for PCO(S). The ROC curve analysis (FIG. 10) did not show that a combination of GDF9 and AMH as a ratio led to increased sensitivity and specificity characteristics, above AMH alone. The combined use BMP15 and AMH as a ratio showed high levels of specificity (83%) and sensitivity (81%) for distinguishing PCO(S) patients from non-PCO(S) patients, comparable to the existing test of use of AMH alone (FIG. 12B).

Serum GDF9 Levels are Lower in Endometriosis Patients

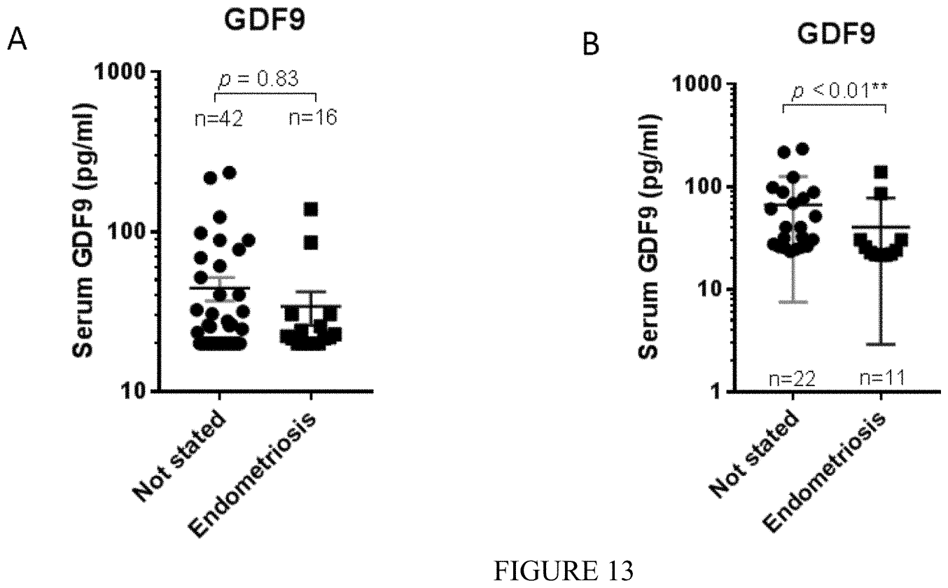

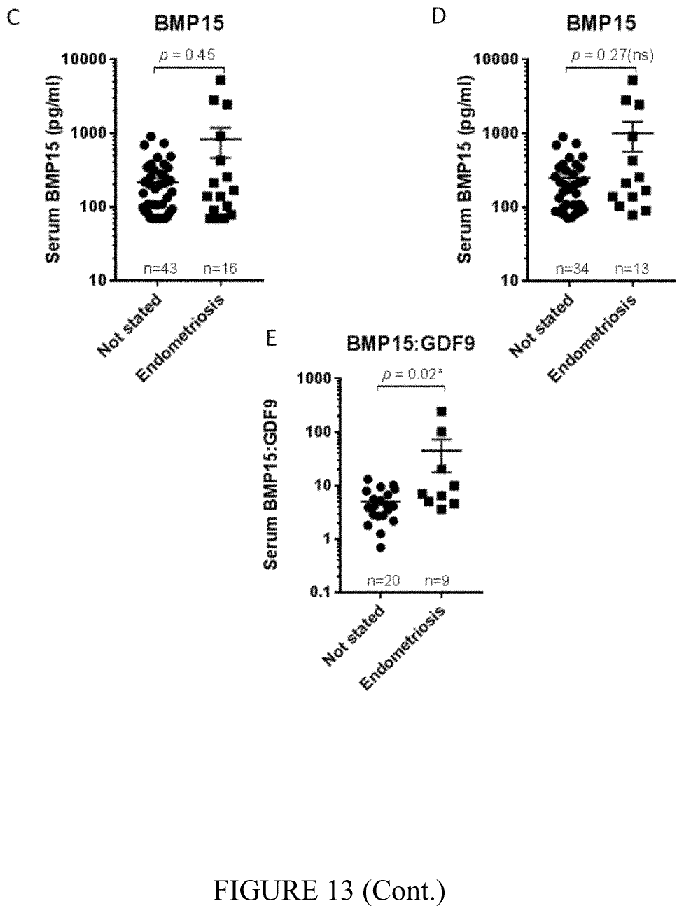

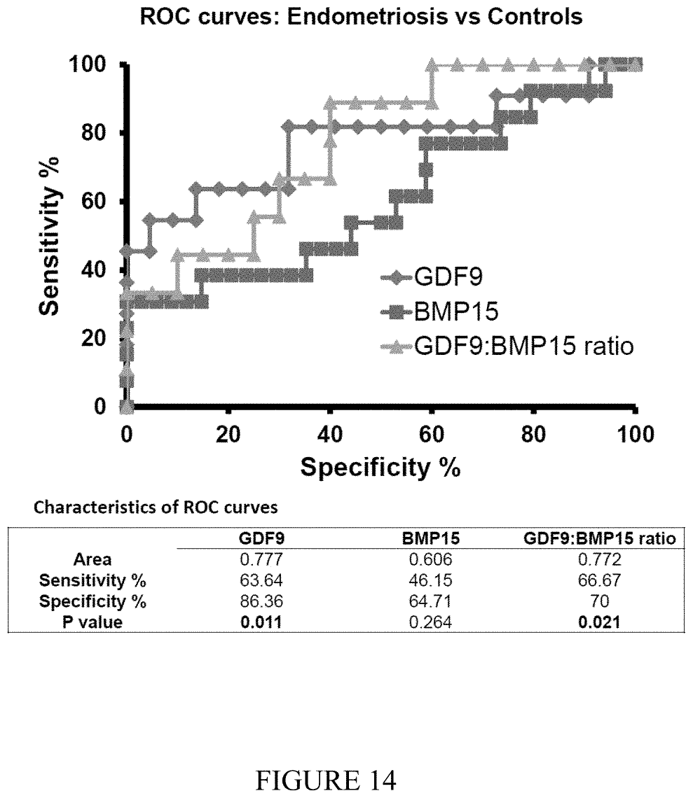

There are currently no reliable serum biomarkers of endometriosis. Furthermore, endometriosis is difficult to diagnose without a laparoscopic surgical procedure. Hence the control group in this study was based on absence of clinical symptoms of endometriosis rather than evidence by laparoscopy. Serum GDF9 levels were significantly lower in endometriosis patients compared with the control group (FIG. 13). Serum BMP15 levels were not different between the two groups (FIG. 13C, 13D). The difficulty with this analysis is that many of the serum GDF9 values were at or below the level of ELISA detection thus a defined level could not be established. A comparison was thus made between those values which were detectable (ie above the level of detection FIGS. 13B, 13D) and the BMP15/GDF9 ratio values for these detectable values determined (FIG. 13E). In both the detectable GDF9 group (FIG. 13B) and BMP15:GDF9 ratio data set (FIG. 13E), significantly lower levels (p=0.01-0.02) were observed in patients with endometriosis. This is reflected in a ROC curve analysis where sensitivity/specificity values of 64%, 86% (respectively) for GDF9 alone and 67%, 70% for GDF9:BMP15 ratio were observed. Development of more sensitive GDF9 ELISAs which can detect lower values (<20 pg/ml) would enable an increased assessment of these clinical groups. AMH levels were not decreased in the endometriosis patients relative to the ANTG group (data not shown).

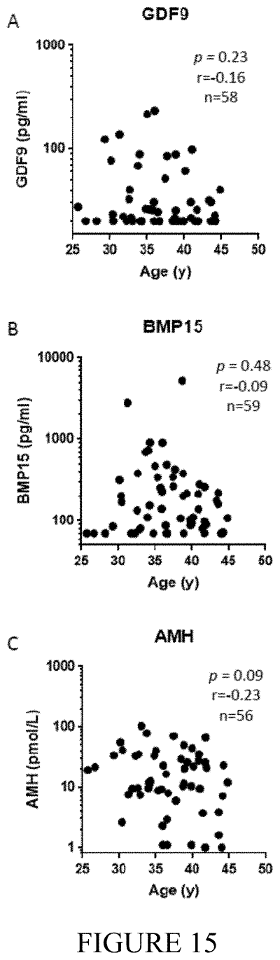

Here the inventors found no clear age-related change with serum GDF9 and BMP15 levels and patient age (25-45 y) (FIG. 15). A comparison in either serum GDF9 or BMP15 <35 vs >35 years showed no significant differences. However, this does not exclude the possibility that these serum hormones levels differ outside this age range.

Serum BMP15 Levels are Stable within Individual Patient Menstrual Cycles and are Unaffected by Ovarian Stimulation

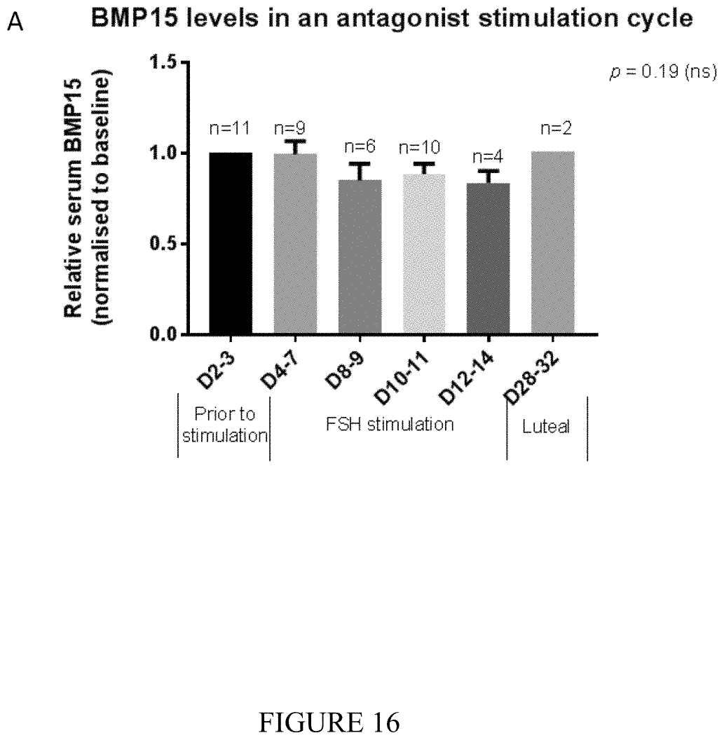

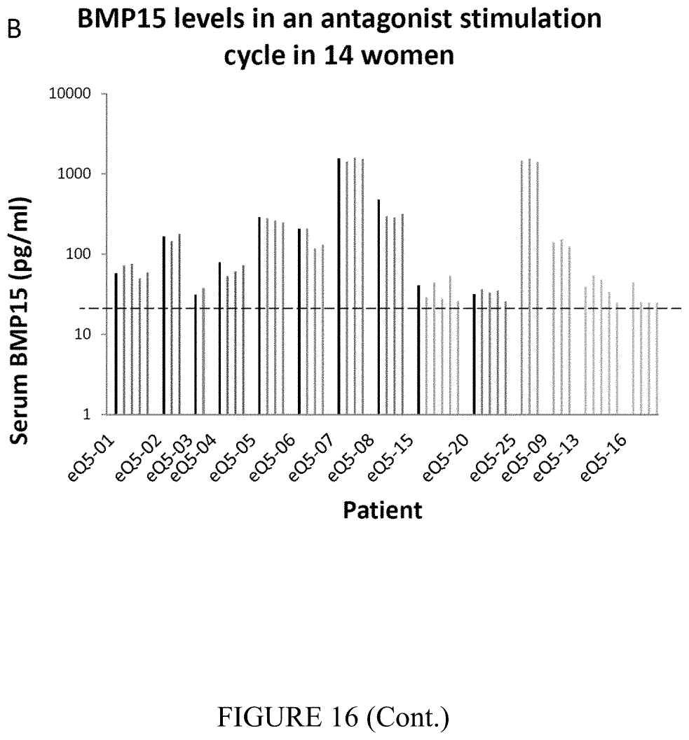

Serum BMP15 was assessed in IVF patients receiving antagonist FSH ovarian stimulation, that had a blood sample prior to stimulation (day 2 or 3), and with multiple (>2) tracked bloods prior to ovulation within the same cycle, following daily FSH injections (FIG. 16). Therefore, analyses included a baseline blood (D2-3), and bloods approximately every 2 days with increasing cumulative FSH dose (stratified as Day 4-7, 8-9, 10-11 and 12-14; FIG. 16A).). The same results but shown as consecutive blood samples for the individual women within a cycle are shown in FIG. 16B. Despite individual women having notable different serum BMP15 levels (FIG. 16B; note log-scale on y-axis), and despite the women receiving different doses of FSH, serum BMP15 levels within patients did not change between the baseline blood values and subsequent bloods post-stimulation. Therefore serum BMP15 levels are stable within individual patient's menstrual cycles, and are not affected by FSH stimulation, regardless of dose, or of a patient's individual natural BMP15 levels.

Serum GDF9 in Male Serum Inversely Correlates with Semen Quality

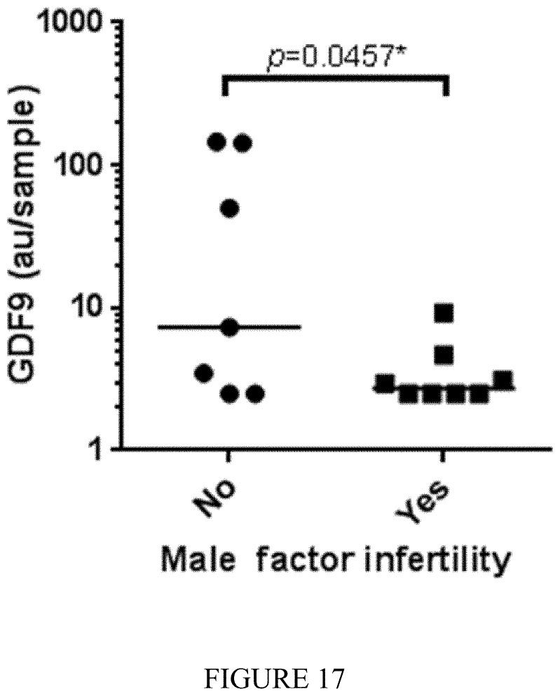

Serum GDF9 levels of 15 males were assessed relative to their semen analysis. Patients with abnormal semen analysis, including reduced motility and abnormal morphology were found to have significantly lower serum GDF9 levels than males with normal semen analyses (p<0.05; FIG. 17).

Application of GDF9 and BMP15 ELISAs to Human Ovarian Cumulus Cell (CC) Extracts

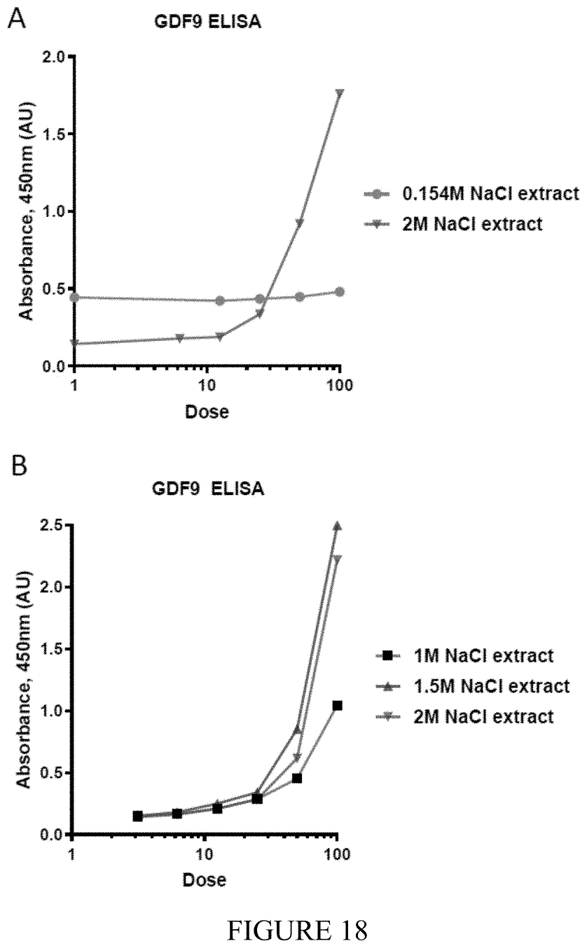

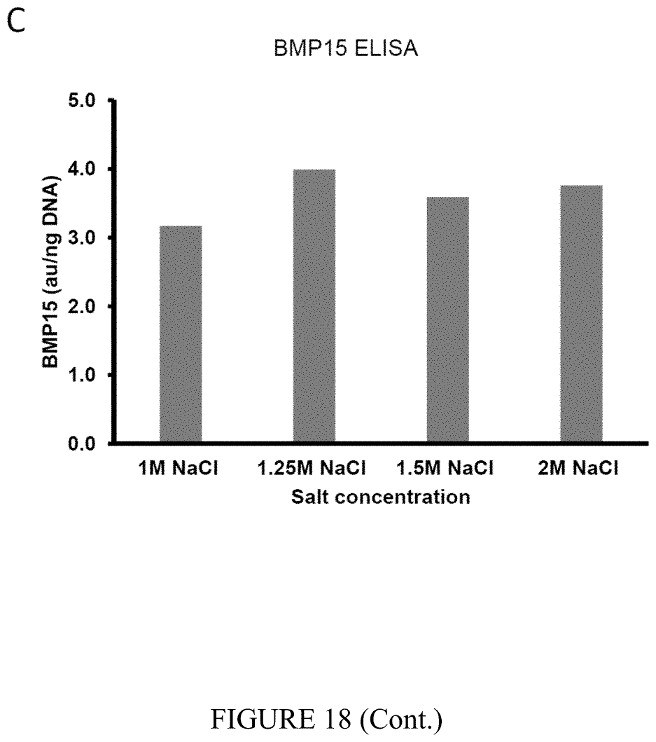

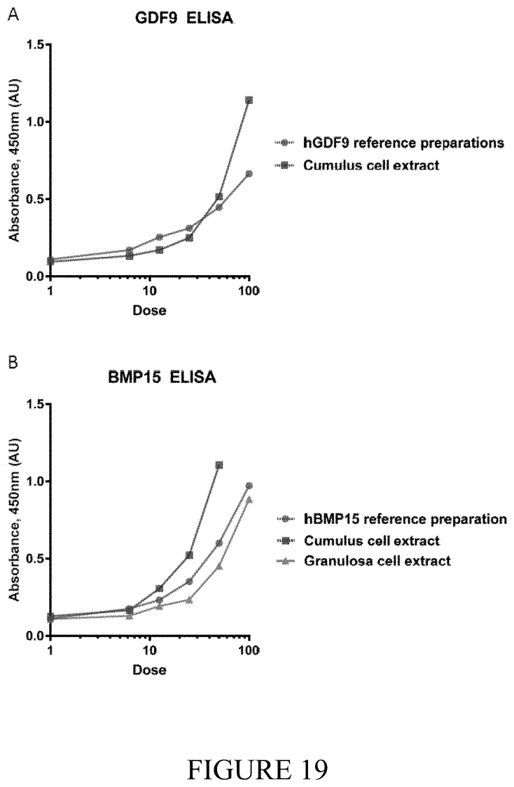

GDF9 and BMP15 are secreted by oocytes and are captured by CCs. CCs do not express or secrete GDF9 and BMP15. Hence, GDF9 and BMP15 attached to the surface of CCs will reflect oocyte production of these important growth factors, and may be useful as diagnostic markers of oocyte quality. Extraction of GDF9 and BMP15 from the cumulus cells was optimised using a buffer containing 1.5M NaCl. Using lower concentrations (0.15M) led to no extraction GDF9 (FIG. 18, BMP15, not shown), while 1M NaCl gave intermediate extractions. Serial dilution of CC extracts in the GDF9 and BMP15 ELISAs gave dose response curves that were not parallel to their respective purified recombinant GDF9 and BMP15 reference preparations (FIG. 19). It is unclear why there is non-parallelism however it is likely a reflection of differing forms of the native GDF9 and BMP15 in the CC extracts, compared to the recombinant preparations. No technical explanation could be identified to explain this observation. Thus, in the BMP15 ELISA, where more work has been done, a human ovarian granulosa cell (GC) extract obtained using the same salt extraction procedure used for the CCs was used as a reference preparation in this ELISA with a defined arbitrary unitage. This GC preparation gave a parallel response with the CC extracts in the BMP15 ELISA (FIG. 19B). A large extract pool was prepared and stored in aliquots at −80 C with one aliquot used/ELISA.

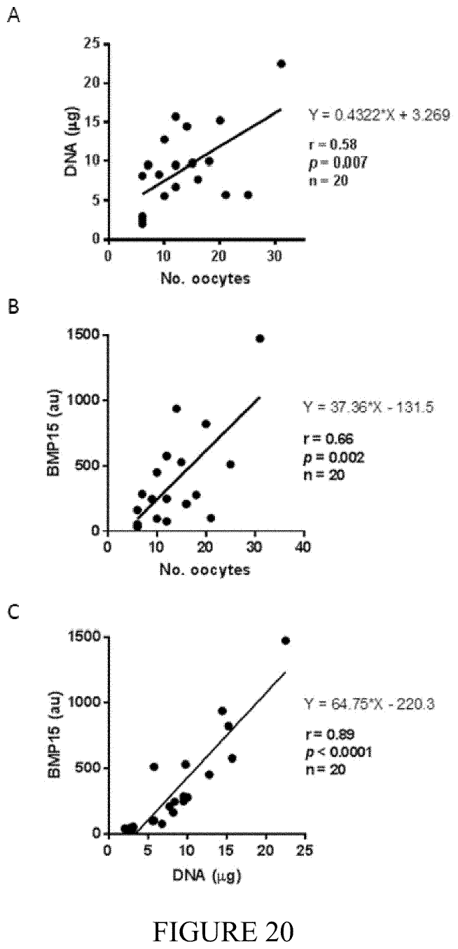

As part of the method validation, a linear response was observed between BMP15 levels and number of oocytes/dish with a correlation coefficient of 0.66, (p=0.002, FIG. 20B). However, it is recognised that the number of CCs/oocyte varies widely (r=0.58, FIG. 20A), attributed mostly to collection procedures: variable effectiveness of recoveries of the cumulus-oocyte complex from differing ovarian follicles, and further exacerbated by subtle differences in collection procedures by differing surgeons during oocyte collection procedure. Hence, there is a need to express BMP15 levels by CC number, rather than per oocyte number. When the BMP15 levels are calibrated in terms of DNA levels for each CC collection, a closer relationship was observed (FIG. 20C; r=0.89, p<0.0001). For the subsequent analyses, BMP15 CC levels were expressed in terms of both their total DNA content and total oocyte dish content.

Cumulus Cell BMP15 Levels Correlate with the Number and Quality of Eggs Retrieved During IVF

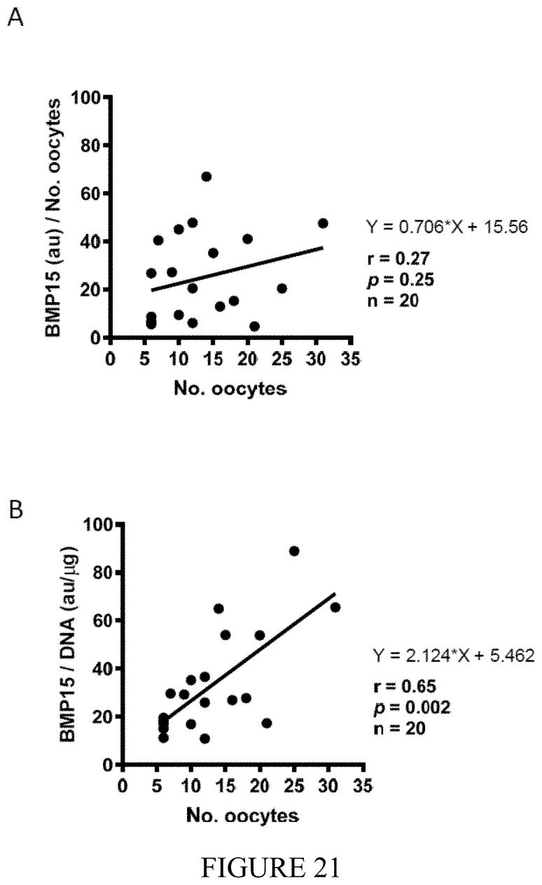

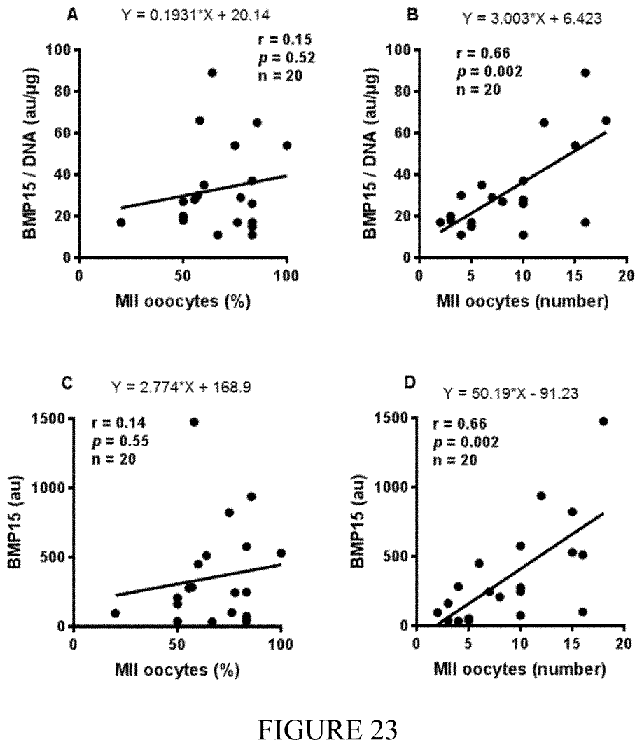

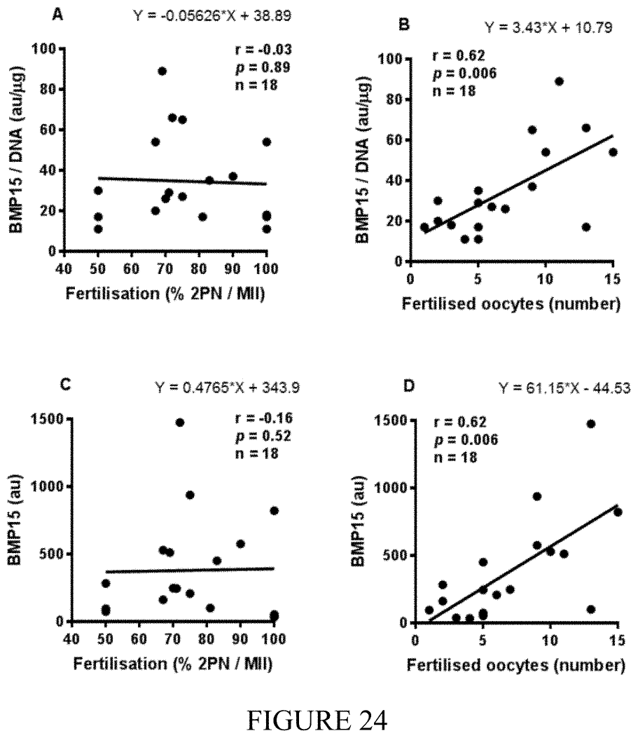

20 individual women undergoing IVF were investigated. BMP15 levels were measured in individuals from the pool of the patient's CCs from all of her oocytes collected on a given day. As expected, patients with more oocytes had more BMP15 in total (FIG. 20B), as also reflected in those patients having more total CC DNA (FIG. 20A). However, there was also a significant positive correlation between BMP15/ug CC DNA and increasing oocyte number (FIG. 21B; 1=0.65, p=0.002). This means that better prognosis patients with more oocytes collected also have more BMP15 per CC, reflecting more BMP15 produced per oocyte. Moreover, patient's total CC BMP15 amounts and BMP15/CC were correlated with the number of mature oocytes (MII oocytes; FIGS. 23D and 23B, respectively) and the number of fertilized oocytes (FIGS. 24D and 24B, respectively). These data indicate that individual oocyte secretion of BMP15 is higher in patients with more oocytes and in patients with more fertilised embryos.

Oocyte-Secretion of BMP15 Declines with Patient Age

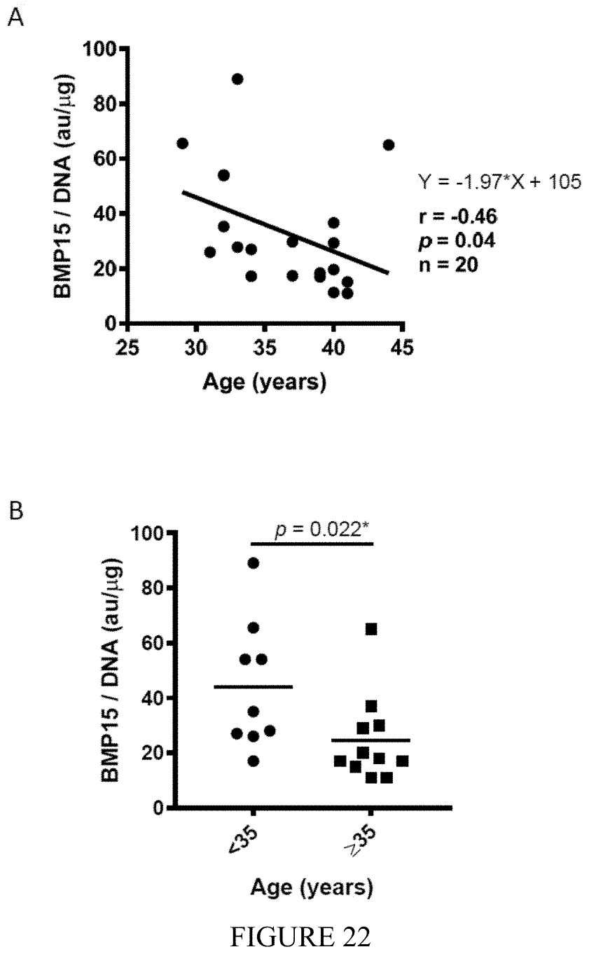

A significant (p=0.04) inverse relationship between BMP15/CC and age was noted (FIG. 22A) with a significant fall (p=0.02) observed in CCs of women >35 compared to <35 years (FIG. 22B).

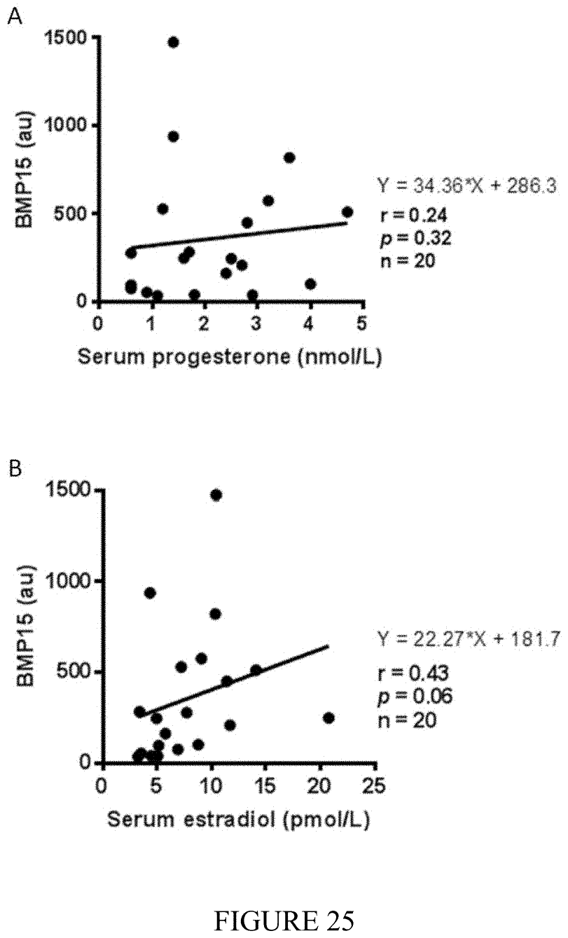

These observations (BMP15/CC correlations with oocyte number, oocyte quality and patient age) parallel the higher pregnancy success rate observed in women with high follicle count and with women of younger age, supporting the claim that BMP15 CC levels may be diagnostic of IVF treatment success. A significant relationship between serum progesterone and total CC BMP15 from the same patient was not evident (FIG. 25A), although there was a strong trend (p=0.06; FIG. 25B) for total CC BMP15 and serum estradiol levels.

### Discussion

The present inventors are the first to describe and validate a series of ELISAs specifically designed to measure GDF9, BMP15 and cumulin in human serum/plasma and from human cells collected during IVF/ICSI. The capacity to detect these growth factors in serum is unexpected as these are local paracrine growth factors, principally secreted by oocytes and spermatocytes only, with no known endocrine function. This first demonstration of the capacity to measure oocyte-secreted biomarkers in serum/plasma enables the application of assays useful in the diagnosis and treatment of reproductive disease including infertility.

The present inventors have demonstrated for the first time that serum GDF9 and BMP15 are markers of ovarian reproductive reserve, comparable in some respects to that seen with AMH, which is the current standard clinical measure of ovarian reserve. Serum GDF9 correlates strongly with oocyte number retrieved in non-PCOS patients. Serum GDF9 levels may prove useful to diagnose a woman's fertility potential, when used in isolation or in combination with serum AMH and other reproductive hormones. As GDF9/BMP15/cumulin are produced by the oocyte only, whereas AMH is not produced by the oocyte (but rather by the oocyte's neighbouring somatic cells), it can be anticipated that measuring serum GDF9/BMP15/cumulin will provide novel physiological insights and thereby complement the diagnostic utility of measuring AMH. As such, in certain clinical scenarios, the combined use of GDF9/BMP15/cumulin with AMH may provide diagnostic clarity not provided by AMH alone.

Just as AMH functions aberrantly in PCO(S) patients compared to non-PCO(S) patients and does not predict oocyte yield in PCO(S) patients, likewise GDF9 and BMP15 did not predict oocyte yield in PCO(S) patients. Combined use of serum BMP15 and AMH levels, or BMP15 with other current diagnostic measures (serum testosterone, antral follicle count, oligomenorrhea) may be useful in diagnosing PCO(S) and also distinguishing differing PCO(S) sub-types not detected by existing diagnostic criteria.

Currently there are no serum/plasma based markers of endometriosis despite the great clinical need. The negligible levels of serum GDF9 seen in patients with endometriosis indicates that GDF9 can be used in a diagnostic assay with significant applications for the diagnosis and treatment management of this common disease.

Serum GDF9, BMP15 and cumulin levels have not previously been described in males. The low levels of serum GDF9 in men with poor semen analyses indicates that these blood-based diagnostics have application in the diagnosis and treatment management of male-factor infertility and other male reproductive diseases.

Investigations examining the levels of BMP15 and GDF9 in cumulus cells from individual patients and moreover from individual oocytes from patients is potentially a useful diagnostic of IVF outcome. GDF9 and BMP15 have previously been crudely measured (principally by Western blot) from cumulus cell and granulosa cell samples discarded from IVF patients. Western blots do not provide an accurate or reliable measure of protein quantification, whereas the ELISAs developed in the current invention provide for the first time the capacity to reliably and accurately quantitate BMP15 and GDF9 levels in human cumulus and granulosa cells discarded during IVF. BMP15 levels expressed per CC DNA show higher levels at a younger age, in patients with higher oocyte number, those with more mature oocytes and more resulting embryos (successful oocyte fertilisation). It is reasonable to expect that this method will be useful in predicting outcomes in women with additional fertility difficulties such as endometriosis and polycystic ovarian disease.

It can also be expected that measurement of BMP15, GDF9 and/or cumulin secreted by individual oocytes, will prove a useful diagnostic indicator of said oocyte's health and developmental potential. Embryo health and hence the probability of pregnancy success is principally determined by oocyte health. Hence, an oocyte quality diagnostic will be useful for diagnosing embryo health and pregnancy potential, and will thereby assist in the management of a patient's IVF cycle. There is great clinical need for such a diagnostic measure of oocyte and embryo health. When women have an oocyte collection procedure for IVF, multiple oocytes are collected (typically 10-15 oocytes, range: 0-30). Currently there is no reliable means to distinguish good from poor quality oocytes/embryos, from the pool of oocytes collected in a patient's IVF cycle. Hence, women commonly receive poor quality embryos transferred back to their uterus which do not lead to a successful pregnancy. This necessitates multiple rounds of embryo transfer of embryos of unknown quality in the hope of generating a successful pregnancy. The capacity to use BMP15, GDF9 and/or cumulin secreted by individual oocytes as a diagnostic measure of individual oocyte/embryo health would improve the efficiency of the IVF process, reduce time to pregnancy success, reduce patient drop-out rates, and reduce cost to patients and health care providers.

Prior to this invention there has been no cumulin assay and cumulin has not previously been measured in such biological sample types. With the current development of a validated ELISA to measure cumulin in complex biological fluids, a simple adaptation of our existing ELISA will enable the measurement of cumulin in cumulus cells, granulosa cells, follicular fluid, and related biologicals that are routinely discarded in an IVF treatment cycle. Hence, measuring cumulin in said samples will provide a valuable non-invasive diagnostic tool of oocyte health, and a diagnostic tool of oocyte health in relation to differing reproductive pathologies (e.g PCO(S), endometriosis).

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Gilchrist et al (2004) Biology of Reproduction, 71:732-739
Gilchrist et al. (2008) Hum Reprod Update, 14(2):159-157
Mottershead et al. (2013) Proc Natl Acad Sci USA, 110:E2257
Mottershead et al. (2015) J Biol Chem, 290(39):24007-24020
Shi et al. (2011) Nature, 474:343-349
Simpson et al. (2014) J Clin Endocrinol Metab, 99(4):E615-24

1. A method for predicting the fertility potential of a subject, the method comprising determining the level of one or more of GDF9, BMP15 and/or cumulin in the subject.

2. The method of claim 1, wherein the level of GDF9, BMP15 and/or cumulin is indicative of oocyte quality or oocyte quantity.

3-7. (canceled)

8. The method of claim 1, wherein the subject is undergoing fertility treatment.

9-10. (canceled)

11. The method of claim 1, wherein the level of GDF9, BMP15 and/or cumulin is determined in a sample obtained from the subject.

12. The method of claim 11, wherein the sample comprises serum, plasma, urine, semen, follicular fluid, somatic cells, culture medium conditioned by an oocyte or embryo, and/or biological material collected during IVF or ICSI treatment.

13-16. (canceled)

17. The method of claim 11, wherein the subject is a PCO(S) patient undergoing OI, IUI, ICSI, IVF, IVM, FET or other assisted reproductive technology and the method comprises determining the level of BMP15.

18-26. (canceled)

27. A method of performing Ovulation Induction (OI), In Vitro Fertilisation (IVF) treatment, Intra-cytoplasmic Sperm Injection (ICSI) treatment, Intrauterine Insemination (IUI) In Vitro Maturation (IVM); frozen embryo transfer (FET) or other assisted reproductive technology on a patient, the method comprising:
i) determining the level of GDF9, BMP15 and/or cumulin in the patient, and
ii) modifying the course of treatment of the OI, IVF, ICSI, or IUI based on the level of GDF9, BMP15 and/or cumulin in the patient;
wherein the method comprises determining the level GDF9, BMP15 and/or cumulin in a sample obtained from the patient.

28-30. (canceled)

31. The method of claim 27, wherein the sample is serum, plasma, semen, urine, follicular fluid, somatic cells, culture medium conditioned by an oocyte or embryo, and/or biological material collected during IVF treatment.

32. The method of claim 31, wherein the follicular fluid and/or somatic cells are collected during IVF treatment.

33. The method of claim 27, wherein the level of GDF9, BMP15 and/or cumulin is determined by ELISA assay.

34. A kit, assay or device for determining the level of GDF9, BMP15 and/or cumulin in a patient sample, the kit assay or device comprising one or more reagents to detect GDF9, BMP15 and/or cumulin in the sample, wherein the sample is selected from serum, plasma, follicular fluid, somatic cells, and/or biological material collected during IVF treatment.

35-41. (canceled)
